(19) United States
(12) Patent Application Publication
(10) Pub. No.: US 2026/0102205 A1
(43) Pub. Date: Apr. 16, 2026

(54) EMITTER STATION INCLUDING SINGLE-PIECE STATION HOUSING FOR USE WITHIN AN INTRAVASCULAR LITHOTRIPSY DEVICE

(71) Applicant: BOLT MEDICAL, INC., Carlsbad, CA (US)

(72) Inventors: Eric Schultheis, San Clemente, CA (US); Joshua D. Huffer, Fallbrook, CA (US); Nelson To, Vista, CA (US); Melody Muehlbauer, Vista, CA (US); Megan Friedlander, Carlsbad, CA (US); Peter Dahl, Riverside, CA (US); Swathi Rangarajan, San Marcos, CA (US)

(73) Assignee: BOLT MEDICAL, INC., Carlsbad, CA (US)

(21) Appl. No.: 19/355,453

(22) Filed: Oct. 10, 2025

Related U.S. Application Data
(60) Provisional application No. 63/705,953, filed on Oct. 10, 2024.
(60) Provisional application No. 63/801,511, filed on May 7, 2025.

(51) Int. Cl. A61B 18/24; A61B 18/00; A61B 18/26

(52) U.S. Cl. CPC A61B 18/245; A61B 18/26; A61B 2018/00351; A61B 2018/263

(57) ABSTRACT

A catheter system configured to use a guidewire lumen, includes an energy source, a first energy guide, and a first emitter station including a first station housing. The energy source generates first energy. The first energy guide receives the first energy from the energy source. The first energy guide has a first guide distal end. The first station housing has (a) a first housing base configured to be secured at least partially about the guidewire lumen, (b) a first guide retainer that extends away from the first housing base, the first guide retainer being configured to selectively receive and retain the first guide distal end of the first energy guide, and (c) a first corresponding plasma target that is spaced apart from the first guide retainer. The first housing base, the first guide retainer, and the first corresponding plasma target are integrally formed with one another.

### RELATED APPLICATION

This Application claims the benefit of priority under 35 U.S.C. § 119 to U.S. Provisional Patent Application No. 63/801,511, filed May 7, 2025, and U.S. Provisional Application No. 63/705,953 filed on Oct. 10, 2024, the entire disclosure of which is hereby incorporated by reference herein for all purposes.

### BACKGROUND

Vascular lesions at a treatment site within and/or adjacent to vessels in the body of a patient can be associated with an increased risk for major adverse events, such as myocardial infarction, embolism, deep vein thrombosis, stroke, and the like. Severe vascular lesions, such as severely calcified vascular lesions, can be difficult to treat and achieve patency for a physician in a clinical setting.

Vascular lesions may be treated using interventions such as drug therapy, balloon angioplasty, atherectomy, stent placement, and vascular graft bypass, to name a few. Such interventions may not always be ideal or may require subsequent treatment to address the lesion.

Intravascular lithotripsy is one method that has been recently used with some success for breaking up vascular lesions within vessels in the body. Intravascular lithotripsy utilizes a combination of pressure waves and bubble dynamics that are generated intravascularly in a fluid-filled balloon catheter. In particular, in certain implementations of an intravascular lithotripsy treatment, a high energy source is used to provide energy to an energy guide and/or an emitter so as to generate plasma and ultimately pressure waves within a fluid-filled balloon in order to crack calcification at a treatment site within the vasculature that includes one or more vascular lesions. Rapid bubble formation from the plasma initiation and resulting localized fluid velocity within the balloon transfers mechanical energy through the incompressible fluid to impart a fracture force on the intravascular calcium, which is opposed to the balloon wall. The rapid change in fluid momentum upon hitting the balloon wall is known as hydraulic shock, or water hammer.

In some embodiments, the intravascular lithotripsy device includes an emitter system that can include one or more emitter stations that are located at different longitudinal positions within the balloon, with each emitter station including one or more individual emitters. Each emitter is constructed and provided independently of the other emitters, with each emitter including at least a portion of an energy guide. Each emitter can be positioned generally adjacent to a guidewire lumen, such as within a groove formed into an outer surface of the guidewire lumen. The emitter can then be secured to the guidewire lumen, such as within the groove, by a combination of heat shrink and adhesive. Unfortunately, assembly of this design can be very tedious and time-consuming due to the small size of the components, and due to the requirement of managing multiple emitters and/or energy guides. Additionally, producing multiple emitter components can also be expensive since each emitter is machined independently using technology such as electric discharge machining (EDM), which requires multiple machines and intricate fixturing and/or tooling to hold the individual emitter components.

Thus, it is desired to modify the design of the emitter system and/or the individual emitter stations so that assembly is simpler, less time-consuming, and less expensive.

### SUMMARY

The present invention is directed toward a catheter system for placement within a blood vessel having a vessel wall or within a heart valve within a body of a patient. The catheter system can be used for treating a treatment site within or adjacent to the vessel wall of the blood vessel, or within or adjacent to the heart valve, within the body of the patient. The catheter system can be configured to use a guidewire lumen. In various embodiments, the catheter system includes an energy source, a first energy guide, and a first emitter station including a first station housing. The energy source generates first energy. The first energy guide receives the first energy from the energy source. The first energy guide has a first guide distal end. The first station housing has (a) a first housing base that is configured to be secured at least partially about the guidewire lumen, (b) a first guide retainer that extends away from the first housing base, the first guide retainer being configured to selectively receive and retain the first guide distal end of the first energy guide, and (c) a first corresponding plasma target that is spaced apart from the first guide retainer. The first housing base, the first guide retainer, and the first corresponding plasma target are integrally formed with one another.

In certain embodiments, the first housing base is at least partially cylinder-shaped and defines an at least partially cylinder-shaped base aperture that extends therethrough, and the guidewire lumen is configured to be positioned at least partially within the base aperture.

In some embodiments, the first housing base is substantially fully cylinder-shaped and defines a substantially fully cylinder-shaped base aperture that extends therethrough; and the guidewire lumen is configured to extend through the base aperture.

In other embodiments, the first housing base includes a base opening that enables the first housing base to be mounted about a side of the guidewire lumen, the base opening being sized such that the first housing base is between approximately 10% and 40% less than fully cylinder-shaped.

In certain embodiments, the first housing base has a substantially smooth inner surface that is configured to be secured to an outer surface of the guidewire lumen.

In some embodiments, the inner surface of the first housing base is secured to the outer surface of the guidewire lumen with an adhesive material.

In certain embodiments, the guidewire lumen is substantially cylinder-shaped; and wherein the outer surface of the guidewire lumen is substantially smooth.

In some embodiments, the first guide retainer extends radially outwardly away from the first housing base.

In other embodiments, the first guide retainer extends radially inwardly away from the first housing base.

In certain embodiments, the first guide retainer is substantially annular-shaped.

In some embodiments, the first guide retainer includes a first annular member and a spaced apart second annular member that are longitudinally aligned with one another; and the first guide distal end of the first energy guide is configured to extend through the first annular member and toward the second annular member to be received and retained within the second annular member.

In other embodiments, the first guide retainer is formed as a groove that extends radially away from the first housing base.

In certain embodiments, the first guide distal end of the first energy guide is configured to be secured within the first guide retainer with an adhesive material.

In many embodiments, the catheter system further includes a first emitter that is incorporated into the first emitter station, the first emitter including (a) the first guide distal end of the first energy guide that is selectively received and retained within the first guide retainer, and (b) the first corresponding plasma target that is spaced apart from the first guide distal end. The first energy guide is configured to emit the first energy in a direction away from the first guide distal end and toward the first corresponding plasma target so that a plasma is generated at the first corresponding plasma target upon receiving the first energy from the first energy guide.

In some embodiments, the first corresponding plasma target has a proximal end that is angled so energy from the plasma generated at the first corresponding plasma target is directed away from the first station housing and toward the treatment site.

In certain embodiments, the catheter system further includes a balloon having a balloon wall that defines a balloon interior, a balloon proximal end, a balloon distal end, and a length that extends from the balloon proximal end to the balloon distal end. In some embodiments, the first emitter is positioned within the balloon interior.

In certain embodiments, the first emitter is positioned at a first longitudinal position within the balloon interior relative to the length of the balloon.

In some embodiments, the energy source further generates second energy. In certain embodiments, the catheter system further includes a second energy guide that receives the second energy from the energy source, the second energy guide having a second guide distal end.

In some embodiments, the first station housing further includes a second guide retainer that extends away from the first housing base, the second guide retainer being configured to selectively receive and retain the second guide distal end of the second energy guide.

In certain embodiments, the catheter system further includes a second emitter that is incorporated into the first emitter station, the second emitter including (a) the second guide distal end of the second energy guide that is selectively received and retained within the second guide retainer, and (b) a second corresponding plasma target that is spaced apart from the second guide distal end. In some embodiments, the second energy guide is configured to emit the second energy in a direction away from the second guide distal end and toward the second corresponding plasma target so that a plasma is generated at the second corresponding plasma target upon receiving the second energy from the second energy guide.

In some embodiments, the energy source further generates third energy. In certain embodiments, the catheter system further includes a third energy guide that receives the third energy from the energy source, the third energy guide having a third guide distal end.

In some embodiments, the first station housing further includes a third guide retainer that extends away from the first housing base, the third guide retainer being configured to selectively receive and retain the third guide distal end of the third energy guide.

In certain embodiments, the catheter system further includes a third emitter that is incorporated into the first emitter station, the third emitter including (a) the third guide distal end of the third energy guide that is selectively received and retained within the third guide retainer, and (b) a third corresponding plasma target that is spaced apart from the third guide distal end. In some embodiments, the third energy guide is configured to emit the third energy in a direction away from the third guide distal end and toward the third corresponding plasma target so that a plasma is generated at the third corresponding plasma target upon receiving the third energy from the third energy guide.

In some embodiments, the energy source further generates fourth energy. In certain embodiments, the catheter system further includes a fourth energy guide that receives the fourth energy from the energy source, the fourth energy guide having a fourth guide distal end.

In certain embodiments, the first station housing further includes a fourth guide retainer that extends away from the first housing base, the fourth guide retainer being configured to selectively receive and retain the fourth guide distal end of the fourth energy guide.

In some embodiments, the catheter system further includes a fourth emitter that is incorporated into the first emitter station, the fourth emitter including (a) the fourth guide distal end of the fourth energy guide that is selectively received and retained within the fourth guide retainer, and (b) a fourth corresponding plasma target that is spaced apart from the fourth guide distal end. In certain embodiments, the fourth energy guide is configured to emit the fourth energy in a direction away from the fourth guide distal end and toward the fourth corresponding plasma target so that a plasma is generated at the fourth corresponding plasma target upon receiving the fourth energy from the fourth energy guide.

In various embodiments, the energy source further generates second energy. In many embodiments, the catheter system further includes a second energy guide that receives the second energy from the energy source, the second energy guide having a second guide distal end; and a second emitter station including a second station housing having (a) a second housing base that is configured to be secured at least partially about the guidewire lumen, (b) a second guide retainer that extends away from the second housing base, the second guide retainer being configured to selectively receive and retain the second guide distal end of the second energy guide, and (c) a second corresponding plasma target that is spaced apart from the second guide retainer, the second housing base, the second guide retainer, and the second corresponding plasma target being integrally formed with one another.

In some embodiments, the catheter system further includes a plurality of first energy guides that are each configured to alternatively receive at least a portion of the first energy from the energy source, each of the plurality of first energy guides including a first guide distal end; and a plurality of second energy guides that are each configured to alternatively receive at least a portion of the second energy from the energy source, each of the plurality of second energy guides including a second guide distal end.

In certain embodiments, the first station housing includes a plurality of first guide retainers that extend away from the first housing base, each of the plurality of first guide retainers being configured to selectively receive and retain the first guide distal end of one of the plurality of first energy guides; and a plurality of first corresponding plasma targets, the first housing base, the plurality of first guide retainers, and the plurality of first corresponding plasma targets being integrally formed with one another.

In some embodiments, the second station housing includes a plurality of second guide retainers that extend away from the second housing base, each of the plurality of second guide retainers being configured to selectively receive and retain the second guide distal end of one of the plurality of second energy guides; and a plurality of second corresponding plasma targets, the second housing base, the plurality of second guide retainers, and the plurality of second corresponding plasma targets being integrally formed with one another.

In many embodiments, the catheter system further includes (a) a first emitter that is incorporated into the first emitter station, the first emitter including the first guide distal end of the first energy guide that is selectively received and retained within the first guide retainer, and the first corresponding plasma target that is spaced apart from the first guide distal end, the first energy guide being configured to emit the first energy in a direction away from the first guide distal end and toward the first corresponding plasma target so that a plasma is generated at the first corresponding plasma target upon receiving the first energy from the first energy guide; and (b) a second emitter that is incorporated into the second emitter station, the second emitter including the second guide distal end of the second energy guide that is selectively received and retained within the second guide retainer, and the second corresponding plasma target that is spaced apart from the second guide distal end, the second energy guide being configured to emit the second energy in a direction away from the second guide distal end and toward the second corresponding plasma target so that a plasma is generated at the second corresponding plasma target upon receiving the second energy from the second energy guide.

In some embodiments, the second emitter included at the second emitter station is rotated about the guidewire lumen relative to the first emitter at the first emitter station.

In various embodiments, the catheter system further includes a balloon having a balloon wall that defines a balloon interior, a balloon proximal end, a balloon distal end, and a length that extends from the balloon proximal end to the balloon distal end; and the first emitter and the second emitter are positioned within the balloon interior.

In certain embodiments, the first emitter is positioned at a first longitudinal position within the balloon interior relative to the length of the balloon; and the second emitter is positioned at a second longitudinal position within the balloon interior relative to the length of the balloon, the second longitudinal position being different than the first longitudinal position.

In some embodiments, the first housing base further includes a groove that is formed along an outer surface of the first housing base; and the second energy guide is positioned within the groove formed along the outer surface of the first housing base as the second energy guide extends past the first housing base and toward the second housing base.

In certain embodiments, the first station housing is formed at least in part from one or more of titanium, stainless steel, tungsten, tantalum, platinum, molybdenum, niobium, and iridium.

In other embodiments, the first station housing is formed at least in part from tungsten.

In still other embodiments, the first station housing is formed at least in part from one or more of a polymer, a polymeric material, a plastic, and nylon.

In various embodiments, the energy source is a laser and the first energy guide is an optical fiber.

The present invention is also directed toward a method for treating a treatment site within or adjacent to a vessel wall of a blood vessel, or within or adjacent to a heart valve, within a body of a patient, including a step of utilizing any embodiments of the catheter system described above.

The present invention is further directed toward a catheter system for treating a treatment site within or adjacent to a vessel wall of a blood vessel, or within or adjacent to a heart valve, within a body of a patient, including an energy source that generates energy; a plurality of energy guides that each selectively receive at least a portion of the energy from the energy source, each of the plurality of energy guides having a guide distal end; and a first emitter station including a first station housing having (a) a first housing base, (b) a plurality of first guide retainers that extend away from the first housing base, each of the plurality of first guide retainers being configured to selectively receive and retain the guide distal end of one of the plurality of energy guides, and (c) a plurality of first corresponding plasma targets that are each positioned spaced apart from one of the plurality of first guide retainers, the first housing base, the plurality of first guide retainers, and the plurality of first corresponding plasma targets being integrally formed with one another.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified schematic cross-sectional view illustration of an embodiment of a catheter system in accordance with various embodiments, the catheter system including an emitter system that includes one or more emitter stations having features of the present invention;

FIG. 2 is a simplified schematic cross-sectional view illustration of a portion of an embodiment of the catheter system illustrated in FIG. 1, including an embodiment of the emitter system with a first emitter station and a second emitter station having features of the present invention;

FIG. 3A is a simplified schematic perspective view illustration of a portion of the catheter system illustrated in FIG. 2, including the first emitter station;

FIG. 3B is a simplified schematic perspective view illustration of another portion of the catheter system illustrated in FIG. 2, including the second emitter station;

FIG. 3C is a simplified schematic perspective view illustration of an embodiment of a station housing that can be included as part of one of the emitter stations illustrated in FIG. 2, and a portion of a plurality of energy guides that have been coupled to the station housing;

FIG. 3D is a simplified schematic perspective view illustration of the station housing illustrated in FIG. 3C;

FIG. 4 is a simplified schematic perspective view illustration of another embodiment of the station housing that can be included as part of one of the emitter stations illustrated in FIG. 2;

FIG. 5 is a simplified schematic perspective view illustration of a portion of another embodiment of the catheter system illustrated in FIG. 1, including another embodiment of the emitter system with a first emitter station and a second emitter station having features of the present invention;

FIG. 6A is a simplified schematic perspective view illustration of an embodiment of the station housing that can be included as part of one of the emitter stations illustrated in FIG. 5;

FIG. 6B is a simplified schematic side view illustration of the station housing illustrated in FIG. 6A;

FIG. 6C is a simplified schematic end view illustration of the station housing illustrated in FIG. 6A;

FIG. 7A is a simplified schematic perspective view illustration of another embodiment of the station housing that could be included as part of one of the emitter stations;

FIG. 7B is a simplified schematic end view illustration of the station housing illustrated in FIG. 7A;

FIG. 8 is a simplified schematic perspective view illustration of a portion of still another embodiment of the catheter system illustrated in FIG. 1, including still another embodiment of the emitter system with a first emitter station, a second emitter station and a third emitter station having features of the present invention;

FIG. 9 is a simplified schematic perspective view illustration of an embodiment of the station housing that can be included as part of one of the emitter stations illustrated in FIG. 8; and

FIG. 10 is a simplified schematic side view illustration of yet another embodiment of the station housing that can be included as part of one of the emitter stations.

While embodiments of the present invention are susceptible to various modifications and alternative forms, specifics thereof have been shown by way of example and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Treatment of vascular lesions at treatment sites within a body of a patient can reduce major adverse events or death in affected subjects. As referred to herein, a major adverse event is one that can occur anywhere within the body due to the presence of a vascular lesion. Major adverse events can include, but are not limited to, major adverse cardiac events, major adverse events in the peripheral or central vasculature, major adverse events in the brain, major adverse events in the musculature, or major adverse events in any of the internal organs.

In various embodiments, the catheter systems and related methods disclosed herein can include a balloon catheter configured to advance to a vascular lesion, such as a calcified vascular lesion or a fibrous vascular lesion, at a treatment site located within a body of a patient. In certain implementations, the “treatment site” can be located at or near a vessel wall of a blood vessel of the patient. Additionally, or in the alternative, in other implementations, the “treatment site” can be at or near a heart valve of the patient. Further, or in the alternative, in still other implementations, the “treatment site” can be at another suitable location within the body of the patient.

As used herein, the terms “treatment site”, “intravascular lesion” and “vascular lesion” may be used interchangeably unless otherwise noted. The intravascular lesions and/or the vascular lesions are sometimes referred to herein as “lesions”.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings. The same or similar nomenclature and/or reference indicators will be used throughout the drawings, and the following detailed description to refer to the same or like parts.

In the interest of clarity, not all of the routine features of the implementations described herein are shown and described. It is appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is recognized that such a development effort might be complex and time-consuming, but would nevertheless be a routine undertaking of engineering for those of ordinary skill in the art having the benefit of this disclosure.

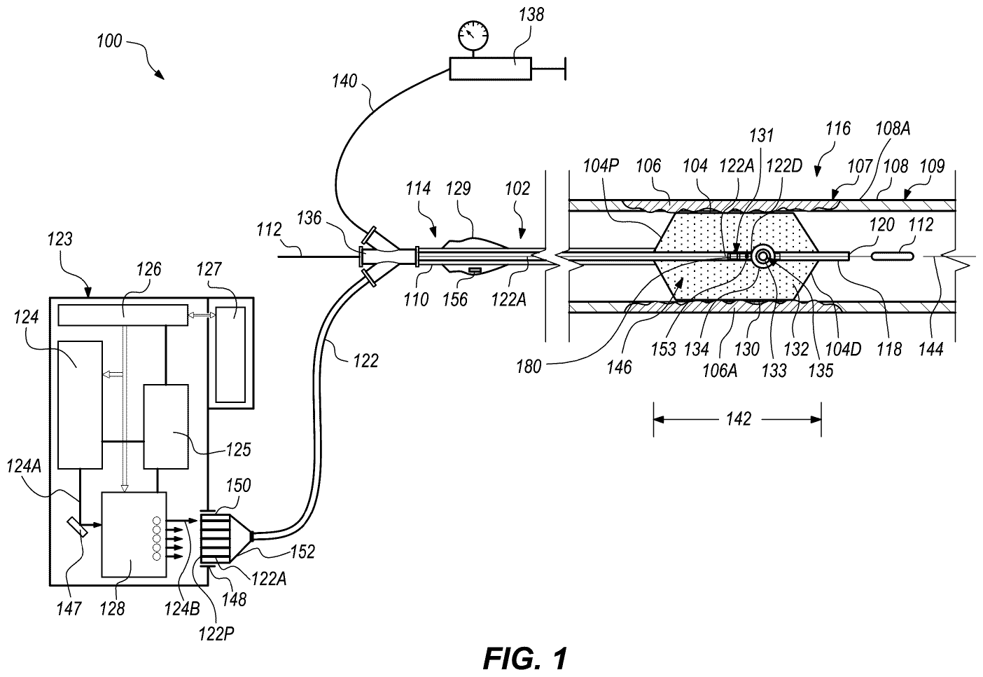

The catheter systems disclosed herein can include many different forms. Referring now to FIG. 1, a simplified schematic cross-sectional view illustration is shown of a catheter system 100 in accordance with various embodiments. The catheter system 100 is suitable for imparting pressure waves to induce fractures at one or more treatment sites 106 within or adjacent to a vessel wall 108A of a blood vessel 108, or on or adjacent to a heart valve, within a body 107 of a patient 109. In the embodiment illustrated in FIG. 1, the catheter system 100 can include (i) a catheter 102 including one or more of an inflatable balloon 104 (sometimes referred to herein simply as a “balloon”), a catheter shaft 110, a guidewire 112, a guidewire lumen 118, an energy guide bundle 122 including one or more energy guides 122A, a source manifold 136, a fluid pump 138, a handle assembly 129, and an emitter system 131; and (ii) a system console 123 including one or more of an energy source 124, a power source 125, a system controller 126, a graphic user interface 127 (a “GUI”), and a multiplexer 128. In various embodiments, the emitter system 131 includes and/or incorporates one or more emitter stations 180, which each include one or more emitters 135 that are configured to direct and/or concentrate energy toward one or more vascular lesions 106A at the treatment site 106 within or adjacent to the vessel wall 108A of the blood vessel 108, or within or adjacent to a heart valve, within the body 107 of the patient 109. Alternatively, the catheter system 100, the catheter 102 and/or the system console 123 can include more components or fewer components than those specifically illustrated and described in relation to FIG. 1.

As illustrated in FIG. 1, the catheter 102 is configured to move to the treatment site 106 within or adjacent to the vessel wall 108A of the blood vessel 108 within the body 107 of the patient 109. The treatment site 106 can include the one or more vascular lesions 106A such as calcified vascular lesions, for example. Additionally, or in the alternative, the treatment site 106 can include vascular lesions 106A such as fibrous vascular lesions. Still alternatively, in some implementations, the catheter 102 can be used at a treatment site 106 within or adjacent to a heart valve within the body 107 of the patient 109. Yet alternatively, in certain implementations, the catheter 102 can be used at a treatment site 106 at another suitable location within the body 107 of the patient 109.

The catheter shaft 110 can extend from a proximal portion 114 of the catheter system 100 to a distal portion 116 of the catheter system 100. The catheter shaft 110 can include a longitudinal axis 144. The guidewire lumen 118 is configured to move over the guidewire 112. As utilized herein, the guidewire lumen 118 defines a conduit through which the guidewire 112 extends. The catheter shaft 110 and/or the catheter 102 can further include an inflation lumen (not shown) and/or various other lumens for various other purposes. In some embodiments, the catheter 102 can have a distal end opening 120 and can accommodate and be tracked over the guidewire 112 as the catheter 102 is moved and positioned at or near the treatment site 106.

The balloon 104 can include a balloon proximal end 104P and a balloon distal end 104D. In certain embodiments, the balloon 104 can be coupled to the catheter shaft 110 and/or to the guidewire lumen 118. More particularly, in some embodiments, the balloon proximal end 104P can be coupled to the catheter shaft 110, and the balloon distal end 104D can be coupled to the guidewire lumen 118.

The balloon 104 includes a balloon wall 130 that defines a balloon interior 146. The balloon 104 is selectively movable between a deflated state and an inflated state (as shown in FIG. 1). More specifically, the balloon 104 can be selectively inflated with a catheter fluid 132 (illustrated as a plurality of small circles) to move from the deflated state, suitable for advancing the catheter 102 through a patient's vasculature, to the inflated state, suitable for anchoring the catheter 102 in position relative to the treatment site 106. Stated in another manner, when the balloon 104 is in the inflated state, at least a portion of the balloon wall 130 of the balloon 104 is configured to be positioned substantially directly adjacent to and/or in contact with the vascular lesions 106A at the treatment site 106.

The balloon 104 suitable for use in the catheter system 100 includes those that can be passed through the vasculature of a patient 109 when in the deflated state. In some embodiments, the balloon 104 is made from silicone. In other embodiments, the balloon 104 can be made from materials such as polydimethylsiloxane (PDMS), polyurethane, polymers such as a polyether block amide (such as PEBAX™) material, nylon, or any other suitable material.

The balloon 104 can have any suitable diameter (in the inflated state). In various embodiments, the balloon 104 can have a diameter (in the inflated state) ranging from less than one millimeter (mm) up to 25 mm. In some embodiments, the balloon 104 can have a diameter (in the inflated state) ranging from at least 1.5 mm up to 14 mm. In other embodiments, the balloon 104 can have a diameter (in the inflated state) ranging from at least two mm up to five mm. Alternatively, the balloon 104 can have a diameter (in the inflated state) that is greater than or less than the ranges specifically noted herein.

In some embodiments, the balloon 104 can have a length 142 ranging from at least three mm to 300 mm. More particularly, in certain embodiments, the balloon 104 can have a length 142 ranging from at least eight mm to 200 mm. It is appreciated that a balloon 104 having a relatively longer length can be positioned adjacent to larger treatment sites 106, and, thus, may be usable for imparting pressure waves onto and inducing fractures in larger vascular lesions 106A or multiple vascular lesions 106A at precise locations within the treatment site 106. It is further appreciated that a longer balloon 104 can also be positioned adjacent to multiple treatment sites 106 at any one given time.

The balloon 104 can be inflated to inflation pressures of between approximately one atmosphere (atm) and 70 atm. In some embodiments, the balloon 104 can be inflated to inflation pressures of from at least 20 atm to 60 atm. In other embodiments, the balloon 104 can be inflated to inflation pressures of from at least six atm to 20 atm. In still other embodiments, the balloon 104 can be inflated to inflation pressures of from at least three atm to 20 atm. In yet other embodiments, the balloon 104 can be inflated to inflation pressures of from at least two atm to ten atm.

The balloon 104 can have various shapes, including, but not to be limited to, a conical shape, a square shape, a rectangular shape, a spherical shape, a conical/square shape, a conical/spherical shape, an extended spherical shape, an oval shape, a tapered shape, a bone shape, a stepped diameter shape, an offset shape, or a conical offset shape.

In some embodiments, the balloon 104 can include a drug eluting coating or a drug eluting stent structure. The drug eluting coating or drug eluting stent can include one or more therapeutic agents including anti-inflammatory agents, anti-neoplastic agents, anti-angiogenic agents, and the like. The drug eluting coating or drug eluting stent can be utilized to further treatment of the treatment site 106 before, during or after any given intravascular lithotripsy procedure.

The catheter fluid 132 used to inflate the balloon 104 can be a liquid or a gas. Some examples of the catheter fluid 132 suitable for use can include, but are not limited to one or more of water, saline, contrast medium, fluorocarbons, perfluorocarbons, gases, such as carbon dioxide, or any other suitable catheter fluid 132. In some embodiments, the catheter fluid 132 can be used as a base inflation fluid. In some embodiments, the catheter fluid 132 can include a mixture of saline to contrast medium in a volume ratio of approximately 50:50. In other embodiments, the catheter fluid 132 can include a mixture of saline to contrast medium in a volume ratio of approximately 25:75. In still other embodiments, the catheter fluid 132 can include a mixture of saline to contrast medium in a volume ratio of approximately 75:25. However, it is understood that any suitable ratio of saline to contrast medium can be used. The catheter fluid 132 can be tailored on the basis of composition, viscosity, and the like so that the generation of plasma and the rate of travel of the acoustic waves and/or pressure waves are appropriately manipulated. In certain embodiments, the catheter fluid 132 suitable for use herein is biocompatible. A volume of catheter fluid 132 can be tailored by the chosen energy source 124 and the type of catheter fluid 132 used.

In some embodiments, the contrast agents used in the contrast media can include, but are not to be limited to, iodine-based contrast agents, such as ionic or non-ionic iodine-based contrast agents. Some non-limiting examples of ionic iodine-based contrast agents include diatrizoate, metrizoate, iothalamate, and ioxaglate. Some non-limiting examples of non-ionic iodine-based contrast agents include iopamidol, iohexol, ioxilan, iopromide, iodixanol, and ioversol. In other embodiments, non-iodine-based contrast agents can be used. Suitable non-iodine containing contrast agents can include gadolinium (III)-based contrast agents. Suitable fluorocarbon and perfluorocarbon agents can include, but are not to be limited to, agents such as the perfluorocarbon dodecafluoropentane (DDFP, C₅F₁₂).

The catheter fluids 132 can include those that include absorptive agents that can selectively absorb light in the ultraviolet region (e.g., at least ten nanometers (nm) to 400 nm), the visible region (e.g., at least 400 nm to 780 nm), or the near-infrared region (e.g., at least 780 nm to 2.5 μm) of the electromagnetic spectrum. Suitable absorptive agents can include those with absorption maxima along the spectrum from at least ten nm to 2.5 μm. Alternatively, the catheter fluids 132 can include those that include absorptive agents that can selectively absorb light in the mid-infrared region (e.g., at least 2.5 μm to 15 μm), or the far-infrared region (e.g., at least 15 μm to one mm) of the electromagnetic spectrum. In various embodiments, the absorptive agent can be those that have an absorption maximum matched with the emission maximum of the laser used in the catheter system 100. By way of non-limiting examples, various lasers usable in the catheter system 100 can include neodymium: yttrium-aluminum-garnet (Nd:YAG—emission maximum=1064 nm) lasers, holmium:YAG (Ho:YAG—emission maximum=2.1 μm) lasers, or erbium:YAG (Er:YAG—emission maximum=2.94 μm) lasers. In some embodiments, the absorptive agents can be water-soluble. In other embodiments, the absorptive agents are not water-soluble. In some embodiments, the absorptive agents used in the catheter fluids 132 can be tailored to match the peak emission of the energy source 124. Various energy sources 124 having emission wavelengths of at least ten nanometers to one millimeter are discussed elsewhere herein.

As an overview, in various embodiments, the catheter system 100 and/or the emitter system 131 can include the one or more emitter stations 180 (and preferably a plurality of emitter stations 180), with each emitter station 180 including one or more emitters 135 (and preferably a plurality of emitters 135). As referred to herein, one or more emitters 135 that are positioned at approximately the same longitudinal position within the balloon interior 146 relative to the length 142 of the balloon 104 can be referred to as an “emitter station”, such as the one or more emitter stations 180 included as part of the emitter system 131 illustrated in FIG. 1.

Each of the emitters 135 is configured to transmit energy from the energy source 124 into the balloon interior 146 in order to generate plasma and/or pressure waves in the catheter fluid 132 within the balloon interior 146. Energy from the plasma and/or pressure waves generated in the catheter fluid 132 within the balloon interior 146 is then used to impart pressure onto and induce fractures in the vascular lesions 106A at the treatment site 106.

More specifically, as illustrated and described herein, each of the emitters 135 includes a guide distal end 122D of one of the energy guides 122A, which is positioned within the balloon interior 146, and a corresponding plasma target 133 (also sometimes referred to as a “plasma generating structure” or a “plasma generator”) that is positioned near, but typically spaced apart from, the guide distal end 122D. Each of the energy guides 122A is configured to selectively receive energy from the energy source 124, under control of the system controller 126 and as directed by the multiplexer 128, guide the energy through the energy guide 122A from a guide proximal end 122P to the guide distal end 122D, and emit the energy from the guide distal end 122D toward the plasma target 133. The energy emitted from the guide distal end 122D impinges upon, contacts, and/or energizes material of the plasma target 133 for purposes of generating plasma in the catheter fluid 132 within the balloon interior 146. The plasma generation ionizes and/or superheats the surrounding catheter fluid 132 and thus causes rapid inertial bubble formation, and imparts pressure waves upon the treatment site 106.

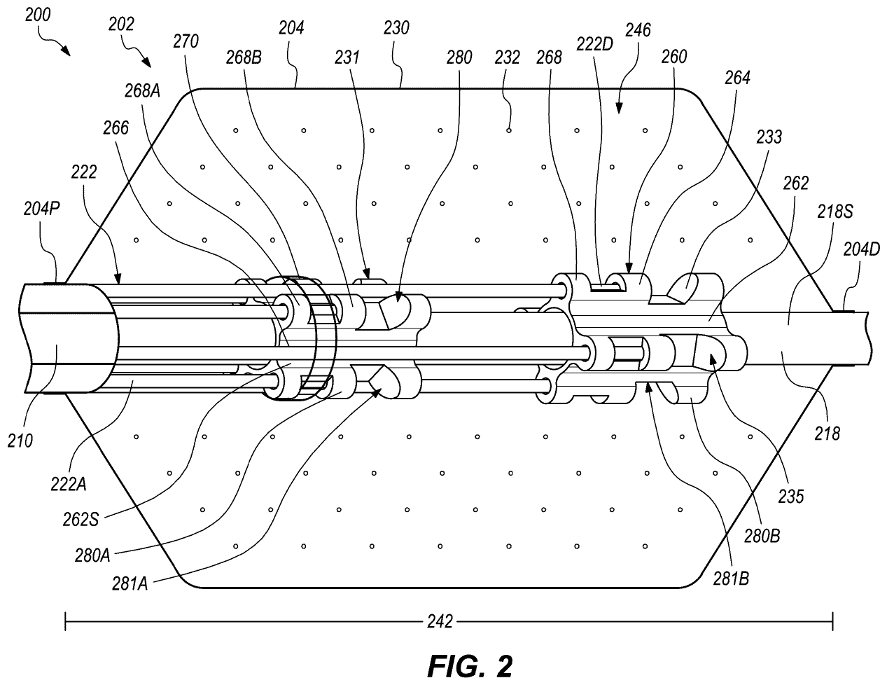

Importantly, in many embodiments, each emitter station 180 includes an integrally-formed, single-piece station housing 260 (illustrated, for example, in FIG. 2) that is configured to maintain a desired positioning between the guide distal end 122D of the energy guide 122A and the corresponding plasma target 133, for each of the emitters 135 included at the emitter station 180. As described in detail herein below in relation to various embodiments, the station housing 260 can have any suitable design and can be manufactured utilizing any suitable manufacturing method.

In certain embodiments, the station housing 260 can include a housing base 262 (illustrated in FIG. 2), one or more (and preferably a plurality of) guide retaining members 264 (illustrated in FIG. 2, and also referred to herein as “guide retainers”) that are each configured to receive and retain at least the guide distal end 122D of one of the energy guides 122A, and one or more (and preferably a plurality of) corresponding plasma targets 133. As described in greater detail herein below, the housing base 262, the one or more guide retainers 264, and the one or more corresponding plasma targets 133, are integrally formed with one another to provide the single-piece station housing 260. Stated in another manner, the station housing 260, and the components incorporated therein, are formed as a unitary structure. With such design, the emitter system 131 and/or the individual emitter stations 180 can be manufactured and assembled in a much simpler, less time-consuming, and less expensive manner than is possible within previous catheter systems. Alternatively, in other embodiments, the housing base 262, the one or more guide retainers 264, and the one or more corresponding plasma targets 133 can be formed as separate components that are secured together to form the single-piece station housing 260.

As so described, in various embodiments, the catheter system 100 is configured to provide a means to power multiple emitter stations 180 in a pressure wave-generating device that is designed to impart pressure onto and induce fractures in vascular lesions 106A, such as calcified vascular lesions and/or fibrous vascular lesions, at the treatment site 106. In many embodiments, the catheter system 100 can be configured and controlled to selectively and/or separately power the multiple emitter stations 180, and/or the multiple emitters 135 within any given emitter station 180, in any desired pattern, order, sequence, and rate of firing.

The catheter shaft 110 of the catheter 102 can be coupled to the plurality of energy guides 122A of the energy guide bundle 122 that are in optical communication with the energy source 124. The energy guide(s) 122A can be disposed along the catheter shaft 110 and within the balloon 104. Each of the energy guides 122A can have the guide distal end 122D that is at any suitable longitudinal position relative to the length 142 of the balloon 104 and/or relative to a length of the guidewire lumen 118. For example, in certain embodiments, a first emitter station 180 can include one or more emitters 135, wherein the guide distal end 122D of each emitter 135 within the first emitter station 180 and the corresponding plasma target 133, even though they can be slightly spaced apart from one another, can be said to be positioned at a first longitudinal position relative to the length 142 of the balloon 104 and/or relative to a length of the guidewire lumen 118; and a second emitter station 180 can include one or more emitters 135, wherein the guide distal end 122D of each emitter 135 within the second emitter station 180 and the corresponding plasma target 133, even though they can be slightly spaced apart from one another, can be said to be positioned at a second longitudinal position relative to the length 142 of the balloon 104 and/or relative to a length of the guidewire lumen 118, with the second longitudinal position being different than the first longitudinal position. It is appreciated that the catheter system 100 can include any suitable or desired number of emitter stations 180 that are each positioned at a different longitudinal position relative to the length 142 of the balloon 104 and/or relative to a length of the guidewire lumen 118. It is further appreciated that each emitter station 180 can include any suitable or desired number of emitters 135, with each emitter 135 within a given emitter station 180 necessarily being at approximately the same longitudinal position relative to the length 142 of the balloon 104 and/or relative to a length of the guidewire lumen 118.

In many embodiments, each energy guide 122A can be an optical fiber and the energy source 124 can be a light source such as a laser. The energy source 124 can be in optical communication with the energy guides 122A at the proximal portion 114 of the catheter system 100. More particularly, the energy source 124 can selectively, simultaneously, sequentially and/or alternatively be in optical communication with each of the energy guides 122A in any desired combination, sequence and/or pattern.

In some embodiments, the catheter shaft 110 can be coupled to multiple energy guides 122A such as a first energy guide, a second energy guide, a third energy guide, etc., which can be disposed at any suitable positions about and/or relative to the guidewire lumen 118 and/or the catheter shaft 110. For example, in certain non-exclusive embodiments, two energy guides 122A can be spaced apart from one another by approximately 180 degrees about the circumference of the guidewire lumen 118 and/or the catheter shaft 110; three energy guides 122A can be spaced apart from one another by approximately 120 degrees about the circumference of the guidewire lumen 118 and/or the catheter shaft 110; four energy guides 122A can be spaced apart from one another by approximately 90 degrees about the circumference of the guidewire lumen 118 and/or the catheter shaft 110; six energy guides 122A can be spaced apart from one another by approximately 60 degrees about the circumference of the guidewire lumen 118 and/or the catheter shaft 110; eight energy guides 122A can be spaced apart from one another by approximately 45 degrees about the circumference of the guidewire lumen 118 and/or the catheter shaft 110; or ten energy guides 122A can be spaced apart from one another by approximately 36 degrees about the circumference of the guidewire lumen 118 and/or the catheter shaft 110. Still alternatively, multiple energy guides 122A need not be uniformly spaced apart from one another about the circumference of the guidewire lumen 118 and/or the catheter shaft 110. More particularly, it is further appreciated that the energy guides 122A can be disposed uniformly or non-uniformly about the guidewire lumen 118 and/or the catheter shaft 110 to achieve the desired effect in the desired locations.

In certain embodiments, the guidewire lumen 118 can be substantially annular-shaped and/or cylindrical-shaped and can have a grooved outer surface, with the grooves extending in a generally longitudinal direction along the guidewire lumen 118. In such embodiments, each of the energy guides 122A and/or the emitter(s) 135 of the emitter system 131 can be positioned, received and retained within an individual groove formed along and/or into the outer surface of the guidewire lumen 118. Alternatively, the guidewire lumen 118 can be formed without a grooved outer surface, and the position of the energy guides 122A and/or the emitter(s) 135 of the emitter system 131 relative to the guidewire lumen 118 can be maintained in another suitable manner. For example, with the design of the station housing 260, the position of the energy guides 122A and/or the emitter(s) 135 of the emitter system 131 relative to the guidewire lumen 118 can be maintained without the need of a grooved outer surface on the guidewire lumen 118.

The catheter system 100, the catheter 102 and/or the energy guide bundle 122 can include any number of energy guides 122A in optical communication with the energy source 124 at the proximal portion 114, and with the catheter fluid 132 within the balloon interior 146 of the balloon 104 at the distal portion 116. For example, in some embodiments, the catheter system 100, the catheter 102 and/or the energy guide bundle 122 can include from one energy guide 122A to greater than 30 energy guides 122A. The guide distal end 122D of each of the energy guides 122A can be at any suitable or desired longitudinal position within the balloon interior 146 relative to the length 142 of the balloon 104 so as to define any suitable or desired number of emitter stations 180. Alternatively, in other embodiments, the catheter system 100, the catheter 102 and/or the energy guide bundle 122 can include greater than 30 energy guides 122A.

The energy guides 122A can have any suitable design that is useful and appropriate for purposes of enabling the generation of plasma and/or pressure waves in the catheter fluid 132 within the balloon interior 146. Thus, the general description of the energy guides 122A as light guides is not intended to be limiting in any manner, except for as set forth in the claims appended hereto. More particularly, although the catheter systems 100 are often described with the energy source 124 as a light source and the one or more energy guides 122A as light guides, the catheter system 100 can alternatively include any suitable energy source 124 and energy guides 122A for purposes of enabling the generation of the desired plasma in the catheter fluid 132 within the balloon interior 146. For example, in one non-exclusive alternative embodiment, the energy source 124 can be configured to provide high-voltage electrical pulses, and each energy guide 122A can include an electrode pair including spaced apart electrodes that extend into the balloon interior 146. In such embodiment, each pulse of high voltage is applied to the electrodes and forms an electrical arc across the electrodes, which, in turn, generates the plasma and forms the pressure waves in the catheter fluid 132 that are utilized to provide the fracture force onto the vascular lesions 106A at the treatment site 106. Still alternatively, the energy source 124 and/or the energy guides 122A can have another suitable design and/or configuration, be it electrical, acoustic, pneumatic, other mechanical, etc.

In certain embodiments, the energy guides 122A can include an optical fiber or flexible light pipe. The energy guides 122A can be thin and flexible and can allow light signals to be sent with very little loss of strength. The energy guides 122A can include a core surrounded by a cladding about its circumference. In some embodiments, the core can be a cylindrical core or a partially cylindrical core. The core and cladding of the energy guides 122A can be formed from one or more materials, including but not limited to one or more types of glass, silica, or one or more polymers. The energy guides 122A may also include a protective coating, such as a polymer. It is appreciated that the index of refraction of the core will be greater than the index of refraction of the cladding.

Each energy guide 122A can guide energy along its length from the guide proximal end 122P toward the guide distal end 122D, with the guide distal end 122D having at least one optical window (not shown) that is positioned within the balloon interior 146.

The energy guides 122A can assume many configurations about and/or relative to the catheter shaft 110 of the catheter 102. In some embodiments, the energy guides 122A can run parallel to the longitudinal axis 144 of the catheter shaft 110. In some embodiments, the energy guides 122A can be physically coupled to the catheter shaft 110. In other embodiments, the energy guides 122A can be disposed along a length of an outer diameter of the catheter shaft 110. In yet other embodiments, the energy guides 122A can be disposed within one or more energy guide lumens within the catheter shaft 110.

The energy guides 122A can also be disposed at any suitable positions about and/or relative to the circumference of the guidewire lumen 118 and/or the catheter shaft 110, and the guide distal end 122D of each of the energy guides 122A can be disposed at any suitable longitudinal position relative to the length 142 of the balloon 104 and/or relative to the length of the guidewire lumen 118 (at any suitable emitter station 180) to more effectively and precisely impart pressure waves for purposes of disrupting the vascular lesions 106A at the treatment site 106.

In certain embodiments, the energy guides 122A can include one or more photoacoustic transducers 153, where each photoacoustic transducer 153 can be in optical communication with the energy guide 122A within which it is disposed. In some embodiments, the photoacoustic transducers 153 can be in optical communication with the guide distal end 122D of the energy guide 122A.

The photoacoustic transducer 153 is configured to convert light energy into an acoustic wave at or near the guide distal end 122D of the energy guide 122A. The direction of the acoustic wave can be tailored by changing an angle of the guide distal end 122D of the energy guide 122A.

In certain embodiments, the photoacoustic transducers 153 can have a shape that corresponds with and/or conforms to the guide distal end 122D of the energy guide 122A. More specifically, In such embodiments, the photoacoustic transducers 153 disposed at the guide distal end 122D of the energy guide 122A can assume the same shape as the guide distal end 122D of the energy guide 122A. For example, in certain non-exclusive embodiments, the photoacoustic transducer 153 and/or the guide distal end 122D can have a conical shape, a convex shape, a concave shape, a bulbous shape, a square shape, a stepped shape, a half-circle shape, an ovoid shape, and the like. The energy guide 122A can further include additional photoacoustic transducers 153 disposed along one or more side surfaces of the length of the energy guide 122A.

In some embodiments, the energy guides 122A, the emitter system 131 and/or the individual emitters 135 can further include one or more diverting structures or “diverters” (not shown in FIG. 1), such as within the energy guide 122A and/or near the guide distal end 122D of the energy guide 122A, that are configured to direct energy from the energy guide 122A toward a side surface which can be located at or near the guide distal end 122D of the energy guide 122A, before the energy is directed toward the balloon wall 130. A diverting structure can include any structure of the system that diverts energy from the energy guide 122A away from its axial path toward a side surface of the energy guide 122A. The energy guides 122A can each include one or more optical windows disposed along the longitudinal or circumferential surfaces of each energy guide 122A and in optical communication with a diverting structure. Stated in another manner, the diverting structures can have any suitable structural configuration that is configured to direct energy in the energy guide 122A toward a side surface that is at or near the guide distal end 122D, where the side surface is in optical communication with an optical window. The optical windows can include a portion of the energy guide 122A that allows energy to exit the energy guide 122A from within the energy guide 122A, such as a portion of the energy guide 122A lacking a cladding material on or about the energy guide 122A.

Examples of the diverting structures suitable for use include a reflecting element, a refracting element, and a fiber diffuser. The diverting structures suitable for focusing energy away from the guide distal end 122D of the energy guide 122A can include, but are not to be limited to, those having a convex surface, a gradient-index (GRIN) lens, and a mirror focus lens. Upon contact with the diverting structure, the energy can be diverted within the energy guide 122A to one or more of the plasma target 133 and the photoacoustic transducer 153 that is in optical communication with a side surface of the energy guide 122A.

When utilized, the plasma target 133 receives energy emitted from the guide distal end 122D of the energy guide 122A to generate plasma in the catheter fluid 132 within the balloon interior 146, which, in turn, causes the creation of plasma bubbles 134 and/or pressure waves that can be directed away from the side surface of the energy guide 122A and toward the balloon wall 130. Additionally, or in the alternative, when utilized, the photoacoustic transducer 153 converts light energy into an acoustic wave that extends away from the side surface of the energy guide 122A.

In certain embodiments, the diverting structures that can be incorporated into the energy guides 122A, can additionally and/or alternatively be incorporated into the design of the emitter system 131 and/or the plasma target(s) 133 for purposes of directing and/or concentrating acoustic and mechanical energy toward specific areas of the balloon wall 130 in contact with the vascular lesions 106A at the treatment site 106 to impart pressure onto and induce fractures in such vascular lesions 106A. It is appreciated that the emitters 135 and/or the plasma target 133 can be designed, positioned and oriented in any suitable manner to direct the acoustic and/or mechanical energy toward any desired portion of the balloon wall 130 that is substantially adjacent to the vascular lesions 106A at the treatment site 106.

The source manifold 136 can be positioned at or near the proximal portion 114 of the catheter system 100. The source manifold 136 can include one or more proximal end openings that can receive the one or more energy guides 122A of the energy guide bundle 122, the guidewire 112, and/or an inflation conduit 140 that is coupled in fluid communication with the fluid pump 138. The catheter system 100 can also include the fluid pump 138 that is configured to inflate the balloon 104 with the catheter fluid 132 as needed.

As noted above, in the embodiment illustrated in FIG. 1, the system console 123 includes one or more of the energy source 124, the power source 125, the system controller 126, the GUI 127, and the multiplexer 128. Alternatively, the system console 123 can include more components or fewer components than those specifically illustrated in FIG. 1. For example, in certain non-exclusive alternative embodiments, the system console 123 can be designed without the GUI 127 and/or the multiplexer 128. Still alternatively, one or more of the energy source 124, the power source 125, the system controller 126, the GUI 127 and the multiplexer 128 can be provided within the catheter system 100 without the specific need for the system console 123.

As shown, the system console 123, and the components included therewith, is operatively coupled to the catheter 102, including the energy guide bundle 122, and the remainder of the catheter system 100. For example, in some embodiments, as illustrated in FIG. 1, the system console 123 can include a console connection aperture 148 (also sometimes referred to generally as a “socket”) by which the energy guide bundle 122 is mechanically coupled to the system console 123. In such embodiments, the energy guide bundle 122 can include a guide coupling housing 150 (which can generally include one or more ferrules) that houses a portion, such as at least the guide proximal end 122P, of each of the energy guides 122A. At least a portion of the guide coupling housing 150 is configured to fit and be selectively retained within the console connection aperture 148 to provide the mechanical coupling between the energy guide bundle 122 and the system console 123, as well as helping to provide an optical coupling between the energy source 124 and each of the energy guides 122A of the energy guide bundle 122.

The energy guide bundle 122 can also include a guide bundler 152 (or “shell”) that brings each of the individual energy guides 122A closer together so that the energy guides 122A and/or the energy guide bundle 122 can be in a more compact form as it extends as part of the catheter 102 into the blood vessel 108 or the heart valve during use of the catheter system 100.

The energy source 124 can be selectively and/or alternatively coupled in optical communication with each of the energy guides 122A, such as to the guide proximal end 122P of each of the energy guides 122A, in the energy guide bundle 122. In particular, the energy source 124 is configured to generate energy in the form of a source beam 124A, such as a pulsed source beam, that can be selectively and/or alternatively directed to and received by each of the energy guides 122A in the energy guide bundle 122. More specifically, in many embodiments, the source beam 124A from the energy source 124 is directed through the multiplexer 128 such that individual guide beams 124B (or “multiplexed beams”) can be selectively and/or alternatively directed into and received by each of the energy guides 122A in the energy guide bundle 122. In particular, each pulse of the energy source 124 and/or each pulse of the source beam 124A can be directed through the multiplexer 128 to generate a separate guide beam 124B that is selectively and/or alternatively directed onto one of the energy guides 122A in the energy guide bundle 122. As such, the energy source 124, through use and/or application of the multiplexer 128, can be utilized to direct energy onto and/or through any of the energy guides 122A, and, thus, to energize any of the emitters 135 at any of the emitter stations 180 that may be included within the catheter system 100. Alternatively, the catheter system 100 can include more than one energy source 124. For example, in one non-exclusive alternative embodiment, the catheter system 100 can include a separate energy source 124 for each of the energy guides 122A in the energy guide bundle 122.

The energy source 124 can have any suitable design. In certain embodiments, the energy source 124 can be configured to provide sub-millisecond pulses of energy from the energy source 124 that are focused onto a small spot in order to couple it into the guide proximal end 122P of the energy guide 122A. Such pulses of energy are then directed and/or guided along the energy guides 122A to a location within the balloon interior 146 of the balloon 104, thereby inducing plasma formation in the catheter fluid 132 within the balloon interior 146 of the balloon 104.

In many embodiments, plasma generation can occur via the plasma target 133, which can include and/or incorporate any suitable structure and can be positioned and oriented in any suitable manner at or near the guide distal end 122D of the energy guide 122A. In particular, the energy emitted from at or near the guide distal end 122D of the energy guide 122A is directed toward and contacts and energizes material of the plasma target 133 for purposes of generating plasma in the catheter fluid 132 within the balloon interior 146. The plasma generation ionizes and superheats the surrounding catheter fluid 132 and thus causes rapid inertial bubble formation, and imparts pressure waves upon the treatment site 106 to provide the fracture force onto the vascular lesions 106A at the treatment site 106. An exemplary plasma-induced bubble 134 is illustrated in FIG. 1.

More specifically, the generation of the plasma bubble 134 creates an outwardly emanating pressure wave throughout the catheter fluid 132 that impacts the balloon wall 130. The impact to the balloon wall 130 causes the balloon 104 to forcefully disrupt and/or fracture the vascular lesions 106A at the treatment site 106. Stated in another manner, the associated rapid formation of the plasma bubble 134 and resulting localized catheter fluid 132 velocity within the balloon 104 transfers mechanical energy though the incompressible catheter fluid 132 to impart a fracture force on the vascular lesions 106A at the treatment site 106. The rapid change in momentum of the catheter fluid 132 upon hitting the balloon wall 130 is known as hydraulic shock, or water hammer. The change in momentum of the catheter fluid 132 is transferred as a fracture force to the vascular lesion 106A which is opposed to the balloon wall 130.

In various non-exclusive alternative embodiments, the sub-millisecond pulses of energy from the energy source 124 can be delivered to the treatment site 106 at a pulse frequency of between approximately one hertz (Hz) and 5000 Hz, between approximately 30 Hz and 1000 Hz, between approximately ten Hz and 100 Hz, or between approximately one Hz and 30 Hz. Alternatively, the sub-millisecond pulses of energy can be delivered to the treatment site 106 at a pulse frequency that can be greater than 5000 Hz or less than one Hz, or any other suitable range of pulse frequencies.

It is appreciated that although the energy source 124 is typically utilized to provide pulses of energy, the energy source 124 can still be described as providing a single source beam 124A, i.e. a single pulsed source beam.

The energy sources 124 suitable for use can include various types of light sources including lasers and lamps. For example, in certain non-exclusive embodiments, the energy source 124 can be an infrared laser that emits energy in the form of pulses of infrared light. Alternatively, as noted above, the energy sources 124 can include any suitable type of energy source.

Suitable lasers can include short pulse lasers on the sub-millisecond timescale. In some embodiments, the energy source 124 can include lasers on the nanosecond (ns) timescale. The lasers can also include short pulse lasers on the picosecond (ps), femtosecond (fs), and microsecond (μs) timescales. It is appreciated that there are many combinations of laser wavelengths, pulse widths and energy levels that can be employed to achieve plasma and the associated cavitation bubbles in the catheter fluid 132 of the catheter 102. In various non-exclusive alternative embodiments, the pulse widths can include those falling within a range including from at least ten ns to 3000 ns, at least 20 ns to 100 ns, or at least one ns to 500 ns. Alternatively, any other suitable pulse width range can be used.

Exemplary nanosecond lasers can include those within the UV to IR spectrum, spanning wavelengths of about ten nanometers (nm) to one millimeter (mm). In some embodiments, the energy sources 124 suitable for use in the catheter systems 100 can include those capable of producing light at wavelengths of from at least 750 nm to 2000 nm. In other embodiments, the energy sources 124 can include those capable of producing light at wavelengths of from at least 700 nm to 3000 nm. In still other embodiments, the energy sources 124 can include those capable of producing light at wavelengths of from at least 100 nm to ten micrometers (μm). Nanosecond lasers can include those having repetition rates of up to 200 kHz.

In some embodiments, the laser can include a Q-switched thulium:yttrium-aluminum-garnet (Tm:YAG) laser. In other embodiments, the laser can include a neodymium:yttrium-aluminum-garnet (Nd:YAG) laser, holmium:yttrium-aluminum-garnet (Ho:YAG) laser, erbium:yttrium-aluminum-garnet (Er:YAG) laser, excimer laser, helium-neon laser, carbon dioxide laser, as well as doped, pulsed, fiber lasers.

In still other embodiments, the energy source 124 can include a plurality of lasers that are grouped together in series. In yet other embodiments, the energy source 124 can include one or more low energy lasers that are fed into a high energy amplifier, such as a master oscillator power amplifier (MOPA). In still yet other embodiments, the energy source 124 can include a plurality of lasers that can be combined in parallel or in series to provide the energy needed to create the plasma bubble(s) 134 in the catheter fluid 132.

The catheter system 100 can generate pressure waves having maximum pressures in the range of at least one megapascal (MPa) to 100 MPa. The maximum pressure generated by a particular catheter system 100 will depend on the energy source 124 and the various parameters of the energy pulses from the energy source 124, the absorbing material, the bubble expansion, the propagation medium, the balloon material, and other factors. In various non-exclusive alternative embodiments, the catheter systems 100 can generate pressure waves having maximum pressures in the range of at least approximately 0.1 MPa to 50 MPa, at least approximately 0.1 MPa to 30 MPa, or at least approximately 15 MPa to 25 MPa.

The pressure waves can be imparted upon the treatment site 106 from a distance within a range from at least approximately 0.1 millimeters (mm) to greater than approximately 25 mm extending radially from the energy guides 122A when the catheter 102 is placed at the treatment site 106. In various non-exclusive alternative embodiments, the pressure waves can be imparted upon the treatment site 106 from a distance within a range from at least approximately ten mm to 20 mm, at least approximately one mm to ten mm, at least approximately 1.5 mm to four mm, or at least approximately 0.1 mm to ten mm extending radially from the energy guides 122A when the catheter 102 is placed at the treatment site 106. In other embodiments, the pressure waves can be imparted upon the treatment site 106 from another suitable distance that is different than the foregoing ranges. In some embodiments, the pressure waves can be imparted upon the treatment site 106 within a range of at least approximately two MPa to 30 MPa at a distance from at least approximately 0.1 mm to ten mm. In some embodiments, the pressure waves can be imparted upon the treatment site 106 from a range of at least approximately two MPa to 25 MPa at a distance from at least approximately 0.1 mm to ten mm. Still alternatively, other suitable pressure ranges and distances can be used.

The power source 125 is electrically coupled to and is configured to provide necessary power to each of the energy source 124, the system controller 126, the GUI 127, the multiplexer 128, and the handle assembly 129. The power source 125 can have any suitable design for such purposes.

The system controller 126 is electrically coupled to and receives power from the power source 125. The system controller 126 is coupled to and is configured to control operation of each of the energy source 124, the GUI 127 and the multiplexer 128. The system controller 126 can include one or more processors or circuits for purposes of controlling the operation of at least the energy source 124, the GUI 127 and the multiplexer 128. For example, the system controller 126 can control the energy source 124 for generating pulses of energy as desired and/or at any desired firing rate. Subsequently, the system controller 126 can then control the multiplexer 128 so that the energy from the energy source 124, as the source beam 124A, can be selectively and/or alternatively directed to each of the energy guides 122A, such as in the form of individual guide beams 124B, in any desired manner.

The system controller 126 can further be configured to control operation of other components or aspects of the catheter system 100, such as the positioning of the catheter 102 and/or the guide distal end 122D of the energy guides 122A adjacent to the treatment site 106, the inflation of the balloon 104 with the catheter fluid 132, etc. Further, or in the alternative, the catheter system 100 can include one or more additional controllers that can be positioned in any suitable manner for purposes of controlling the various operations of the catheter system 100. For example, in certain embodiments, an additional controller and/or a portion of the system controller 126 can be positioned and/or incorporated within the handle assembly 129.

The GUI 127 is accessible by the user or operator of the catheter system 100. The GUI 127 is electrically connected to the system controller 126. With such design, the GUI 127 can be used by the user or operator to ensure that the catheter system 100 is effectively utilized to impart pressure onto and induce fractures into the vascular lesions 106A at the treatment site 106. The GUI 127 can provide the user or operator with information that can be used before, during and after use of the catheter system 100. In one embodiment, the GUI 127 can provide static visual data and/or information to the user or operator. In addition, or in the alternative, the GUI 127 can provide dynamic visual data and/or information to the user or operator, such as video data or any other data that changes over time during use of the catheter system 100. In various embodiments, the GUI 127 can include one or more colors, different sizes, varying brightness, etc., that may act as alerts to the user or operator. Additionally, or in the alternative, the GUI 127 can provide audio data or information to the user or operator. The specifics of the GUI 127 can vary depending upon the design requirements of the catheter system 100, or the specific needs, specifications and/or desires of the user or operator.

The multiplexer 128 is configured to selectively and/or alternatively direct energy from the energy source 124 to each of the energy guides 122A in the energy guide bundle 122. More particularly, the multiplexer 128 is configured to receive energy from the energy source 124, such as in the form of a single source beam 124A from a single laser source, and selectively and/or alternatively direct such energy in the form of individual guide beams 124B, as desired, to each of the energy guides 122A in the energy guide bundle 122. As such, the multiplexer 128 enables a single energy source 124 to be channeled separately in any desired sequence or pattern through a plurality of energy guides 122A such that the catheter system 100 is able to impart pressure onto and induce fractures in vascular lesions 106A at the treatment site 106 within or adjacent to a vessel wall 108A of the blood vessel 108 in a desired manner. As shown, in certain embodiments, the catheter system 100 can include one or more optical elements 147 for purposes of directing the energy, such as the source beam 124A, from the energy source 124 to the multiplexer 128.

The multiplexer 128 can have any suitable design for purposes of selectively and/or alternatively directing the energy from the energy source 124 to each of the energy guides 122A of the energy guide bundle 122.

As shown in FIG. 1, the handle assembly 129 can be positioned at or near the proximal portion 114 of the catheter system 100, and/or near the source manifold 136. In this embodiment, the handle assembly 129 is coupled to the balloon 104 and is positioned spaced apart from the balloon 104. Alternatively, the handle assembly 129 can be positioned at another suitable location.

The handle assembly 129 is attached to the catheter shaft 110 and is handled and used by the user or operator to operate, position and control the catheter 102. The design and specific features of the handle assembly 129 can vary to suit the design requirements of the catheter system 100. In the embodiment illustrated in FIG. 1, the handle assembly 129 is separate from, but in electrical and/or fluid communication with one or more of the system controller 126, the energy source 124, the fluid pump 138, and the GUI 127.

In some embodiments, the handle assembly 129 can integrate and/or include at least a portion of the system controller 126 within an interior of the handle assembly 129. For example, as shown, in certain embodiments, the handle assembly 129 can include circuitry 156, which is electrically coupled between catheter electronics and the system console 123, and which can form at least a portion of the system controller 126. In some embodiments, the circuitry 156 can transmit such electrical signals or otherwise provide data to the system controller 126.

In one embodiment, the circuitry 156 can include a printed circuit board having one or more integrated circuits, or any other suitable circuitry. In an alternative embodiment, the circuitry 156 can be omitted, or can be included within the system controller 126, which in various embodiments can be positioned outside of the handle assembly 129, such as within the system console 123. It is understood that the handle assembly 129 can include fewer or additional components than those specifically illustrated and described herein.

In various embodiments, as noted above, the emitter system 131 includes the one or more emitter stations 180 (and preferably a plurality of emitter stations 180), with each emitter station 180 including the one or more emitters 135 (and preferably a plurality of emitters 135). As further noted above, each emitter 135 includes the guide distal end 122D of one of the energy guides 122A and the corresponding plasma target 133 that is positioned near, but typically spaced apart from, the guide distal end 122D.

Each of the energy guides 122A, upon receiving energy from the energy source 124, is configured to guide and/or transmit the energy from the energy source 124 from the guide proximal end 122P to the guide distal end 122D. The energy is then emitted from at or near the guide distal end 122D into the balloon interior 146, and toward the plasma target 133. The energy impinges on, contacts, and/or energizes the material of the plasma target 133 so that plasma and/or pressure waves are generated in the catheter fluid 132 within the balloon interior 146. Each of the emitters 135 is further configured to direct and/or concentrate energy from the plasma and/or pressure waves generated in the catheter fluid 132 within the balloon interior 146 so as to impart pressure onto and induce fractures in vascular lesions 106A at the treatment site 106 within or adjacent to a vessel wall 108A of a blood vessel 108 or a heart valve.

By positioning the plasma target 133 away from the guide distal end 122D of the energy guide 122A, damage to the energy guide 122A from the generated plasma bubble 134 is less likely to occur than if the plasma bubble 134 was generated at or more proximate the guide distal end 122D of the energy guide 122A. Stated another way, the presence of the plasma target 133, and positioning the plasma target 133 away from the guide distal end 122D of the energy guide 122A, causes the plasma bubble 134 to in turn be generated away from the guide distal end 122D of the energy guide 122A, reducing the likelihood of damage to the energy guide 122A.

As referred to herein, the plasma target 133 can include and/or incorporate any suitable type of structure that is located at or near the guide distal end 122D of the energy guide 122A. For example, in certain embodiments, the plasma target 133 can be provided in the form of a backstop-type structure with an angled face that redirects energy emitted from the guide distal end 122D toward the balloon wall 130 of the balloon 104 and/or toward the vessel wall 108A of the blood vessel 108 at the treatment site 106. Alternatively, the plasma target 133 can have another suitable structural design.

In many embodiments, the present invention utilizes a laser light source or other suitable light source as the energy source 124, and is configured to shine laser light energy onto the plasma target 133 to cause the plasma generation via interaction with a specific plasma target material rather than optical breakdown of the catheter fluid 132. This moves the plasma creation away from the guide distal end 122D of the energy guide 122A (which can be an optical fiber in some embodiments). As noted, this can be accomplished by positioning the plasma target 133 away from the guide distal end 122D of the energy guide 122A to absorb the light energy and convert it into a plasma at some distance away from the guide distal end 122D of the energy guide 122A.

In various embodiments, each emitter station 180 further includes the integrally-formed station housing 260 that is configured to maintain the desired positioning between the guide distal end 122D of each of the energy guides 122A at that emitter station 180, and the corresponding plasma target 133 for each of the energy guides 122A. By effectively maintaining the desired positioning between the guide distal end 122D of the energy guide 122A and the corresponding plasma target 133 for each of the emitters 135, and with the particular design features that may be incorporated into the emitter system 131, such as the integrally-formed station housing 260, the emitter system 131 is configured to concentrate and direct acoustic and/or mechanical energy toward specific areas of the balloon wall 130 in contact with the vascular lesions 106A at the treatment site 106 to enhance the delivery of such energy to the treatment site 106. Thus, the emitter system 131 is able to effectively improve the efficacy of the catheter system 100. The simplicity of the station housing 260, as described in greater detail herein below, further enables the catheter system 100, the emitter system 131, and/or the individual emitter stations 180 to be formed in a manner that is simpler, less time-consuming, and less expensive.

As further noted above, the station housing 260 can include the housing base 262, the one or more (and preferably a plurality of) guide retainers 264 that extend away from the housing base 262 and that are each configured to receive and retain at least the guide distal end 122D of one of the energy guides 122A, and the one or more (and preferably a plurality of) corresponding plasma targets 133. It is appreciated that with the station housing 260 being an integrally-formed, single-piece component, each of the housing base 262, the one or more guide retainers 264, and the one or more corresponding plasma targets 133 of the station housing 260 are formed from the same materials. Alternatively, the station housing 260 can include more components or fewer components than what is specifically illustrated and described herein.

The station housing 260, and thus the housing base 262, the one or more guide retainers 264, and the one or more corresponding plasma targets 133, can be formed from any suitable materials, such that the energy emitted from the guide distal end 122D of the energy guide 122A contacting the plasma target 133 generates the desired plasma in the catheter fluid 132 within the balloon interior 146. For example, in certain non-exclusive embodiments, the station housing 260, and thus the housing base 262, the one or more guide retainers 264, and the one or more corresponding plasma targets 133, can be formed, at least in part, from one or more metals and/or metal alloys having relatively high melting temperatures, such as titanium, stainless steel, tungsten, tantalum, platinum, molybdenum, niobium, iridium, etc. Alternatively, the station housing 260, and thus the housing base 262, the one or more guide retainers 264, and the one or more corresponding plasma targets 133, can be formed from at least one of magnesium oxide, beryllium oxide, tungsten carbide, titanium nitride, titanium carbonitride, and titanium carbide. Still alternatively, the station housing 260, and thus the housing base 262, the one or more guide retainers 264, and the one or more corresponding plasma targets 133, can be formed from at least one of chemical vapor deposition (CVD) diamond and diamond. In other embodiments, the station housing 260, and thus the housing base 262, the one or more guide retainers 264, and the one or more corresponding plasma targets 133, can be formed from a transition metal, an alloy metal, or a ceramic material. Yet alternatively, in some embodiments, the station housing 260, and thus the housing base 262, the one or more guide retainers 264, and the one or more corresponding plasma targets 133, can be formed at least partially from a polymer, a polymeric material, and/or a plastic such as polyimide and nylon. Still alternatively, the station housing 260, and thus the housing base 262, the one or more guide retainers 264, and the one or more corresponding plasma targets 133, can be formed from any other suitable materials.

Further details of various embodiments of the emitter system 131, the emitter stations 180 and/or the individual emitters 135 are illustrated and described in detail herein below within subsequent Figures.

As with all embodiments illustrated and described herein, various structures may be omitted from the figures for clarity and ease of understanding. Additionally, the figures may include certain structures that can be omitted without deviating from the intent and scope of the invention. It is further recognized that the structures included in the various figures shown and described herein are not necessarily drawn to scale for ease of viewing and/or understanding.

FIG. 2 is a simplified schematic cross-sectional view illustration of a portion of an embodiment of the catheter system 200 illustrated in FIG. 1, including an embodiment of the emitter system 231 with a pair of emitter stations 280, such as a first emitter station 280A and a second emitter station 280B, having features of the present invention. As noted above, it is appreciated that the catheter system 200 can include any suitable number of emitter stations 280, which can alternatively be greater than two emitter stations 280, or only a single emitter station 280.

As shown in FIG. 2, the catheter system 200 again includes at least a catheter 202 including a balloon 204, a catheter shaft 210, a guidewire lumen 218, an energy guide bundle 222 including one or more energy guides 222A, and the emitter system 231. The balloon 204, the catheter shaft 210, the guidewire lumen 218, and the energy guides 222A are substantially similar in design and function to what was described in detail herein above. Accordingly, such components will not be described in detail in relation to the embodiment illustrated in FIG. 2. It is further appreciated that certain components of the catheter system 100 illustrated and described above in relation to FIG. 1, such as the guidewire 112, the source manifold 136, the fluid pump 138, the handle assembly 129, the system console 123, the energy source 124, the power source 125, the system controller 126, the GUI 127, and the multiplexer 128, are not illustrated in FIG. 2 for purposes of simplicity and ease of illustration, but would typically be included in many embodiments.

As with the previous embodiment, the catheter 202 is again configured to move to a treatment site 106 (illustrated in FIG. 1) to treat vascular lesions 106A (illustrated in FIG. 1) within or adjacent to the vessel wall 108A (illustrated in FIG. 1) of the blood vessel 108 (illustrated in FIG. 1), or within or adjacent to a heart valve, within the body 107 (illustrated in FIG. 1) of the patient 109 (illustrated in FIG. 1).

In this embodiment, the balloon 204 again includes a balloon proximal end 204P that can be coupled to the catheter shaft 210, and a balloon distal end 204D that can be coupled to the guidewire lumen 218. The balloon 204 again also includes a balloon wall 230 that defines a balloon interior 246, with the balloon 204 being selectively inflatable with a catheter fluid 232 to expand from a deflated state suitable for advancing the catheter 202 through a patient's vasculature, to an inflated state suitable for anchoring the catheter 202 in position relative to the treatment site 106.

The catheter system 200 and/or the catheter 202 can include any desired number of energy guides 222A. For example, in the embodiment illustrated in FIG. 2, the catheter system 200 and/or the catheter 202 includes eight energy guides 222A, with the guide distal end 222D of four of the energy guides 222A being received and retained at the first emitter station 280A, and the guide distal end 222D of another four energy guides 222A being received and retained at the second emitter station 280B. It is appreciated that not all eight energy guides 222A are visible in FIG. 2, as some energy guides 222A are hidden from view behind other components of the catheter system 200. Alternatively, the catheter system 200 and/or the catheter 202 can include greater than eight energy guides 222A or fewer than eight energy guides 222A, and/or a greater or lesser number of energy guides 222A can be received and retained at each of the emitter stations 280A, 280B.

Each of the emitter stations 280A, 280B are positioned at different longitudinal locations relative to the length 242 of the balloon 204. As noted above, in the embodiment shown in FIG. 2, the emitter system 231 includes the first emitter station 280A, which is positioned at a first longitudinal position 281A relative to the length 242 of the balloon 204 and/or relative to the length of the guidewire lumen 218; and the second emitter station 280B, which is positioned at a second longitudinal position 281B relative to the length 242 of the balloon 204 and/or relative to the length of the guidewire lumen 218. As shown, the second longitudinal position 281B is different than the first longitudinal position 281A.

The first emitter station 280A can include any suitable number of emitters 235. More particularly, the first emitter station 280A can be configured to receive and retain the guide distal end 222D of any suitable number of energy guides 222A, which can each be included and/or incorporated into a corresponding emitter 235, and can further include any suitable number of corresponding plasma targets 233 that cooperate with the guide distal end 222D to form each individual emitter 235. In the embodiment illustrated in FIG. 2, the first emitter station 280A includes four individual emitters 235 that are radially spaced apart from one another by approximately ninety degrees. Stated in another manner, as noted above, the first emitter station 280A receives and retains the guide distal end 222D of four energy guides 222A, as well as incorporating four corresponding plasma targets 233, to form the four emitters 235 that are radially spaced apart from one another by approximately ninety degrees.

Similarly, the second emitter station 280B can include any suitable number of emitters 235. More particularly, the second emitter station 280B can be configured to receive and retain any suitable number of energy guides 222A, which can each include a guide distal end 222D that is included and/or incorporated into a corresponding emitter 235, and can further include any suitable number of plasma targets 233 that cooperate with the guide distal end 222D to form each individual emitter 235. In the embodiment illustrated in FIG. 2, the second emitter station 280B also includes four individual emitters 235 that are radially spaced apart from one another by approximately ninety degrees. Stated in another manner, as noted above, the second emitter station 280B receives and retains the guide distal end 222D of another four energy guides 222A, as well as incorporating four corresponding plasma targets 233, to form the four emitters 235 that are radially spaced apart from one another by approximately ninety degrees. As further illustrated in FIG. 2, the emitters 235 included at the second emitter station 280B are rotated approximately 45 degrees relative to the emitters 235 at the first emitter station 280A. The four energy guides 222A whose guide distal ends 222D form part of the four emitters 235 included at the second emitter station 280B are also shown as passing through the first emitter station 280A before extending on to the second emitter station 280B.

The design of the emitter stations 280A, 280B, and the individual emitters 235 within each emitter station 280A, 280B, can be varied to suit the requirements of the catheter system 200. In this embodiment, the design of each emitter station 280A, 280B can be substantially the same as one another. For example, in the embodiment illustrated in FIG. 2, each emitter station 280A, 280B includes a station housing 260 that is utilized to maintain a desired positioning between the guide distal end 222D of the energy guide 222A and the corresponding plasma target 233, for each of the emitters 235 included at the emitter station 280A, 280B. More particularly, since each emitter station 280A, 280B shown in FIG. 2 is configured to include four emitters 235, the station housing 260 is utilized to maintain a desired positioning between the guide distal end 222D of four energy guides 222A and the four corresponding plasma targets 233, for each of the four emitters 235 included at the emitter station 280A, 280B.

In several embodiments, the station housing 260 includes a housing base 262, one or more guide retainers 264 (four guide retainers 264 in this particular embodiment) that extend away from the housing base 262 and that are each configured to receive and retain at least the guide distal end 222D of one of the energy guides 222A, and one or more corresponding plasma targets 233 (four corresponding plasma targets 233 in this particular embodiment).

The station housing 260 can be formed by any suitable method of manufacturing. For example, in some embodiments, the station housing 260 can be manufactured from raw material that is received as a rod stock and that is subsequently formed into the desired shape. In other embodiments, the station housing 260 can be manufactured by utilizing a shaped wire cross-section, having the raw materials in the form of wires drawn and/or extruded to be formed into the desired shape. The process can use shaped wire to simplify downstream manufacturing of multi-emitter stations. This reduces the need to shape the outer profile of the components by electrical discharge machining (EDM) or other means. The shaped wire can be manufactured by wire drawing, metal injection molding (MIMs), or being laser cut. Further, the shaped wires can be drawn with dissolvable cores to provide a channel for lumen(s), guidewires, etc. In still other embodiments, the station housing 260 can be manufactured utilizing a photolithography method, using techniques found in micro-electromechanical systems (MEMs) and the semiconductor industry to produce components out of metal with micrometer-level minimum feature size. In such embodiments, the metallic component can be manufactured in conjunction with a polymer to provide a built-in insulative sleeve. In yet other embodiments, the station housing 260 can be manufactured utilizing composite wire made with a refractory metal core for the plasma target and clad with a higher fracture toughness/ductile exterior to reduce the chance of fragmentation from the plasma event/cavitation forces. The refractory metal center can be as large as the laser spot size. This design would have an external skin that would add mechanical strength to the finished component. The external skin would not be the metal that comes into contact with the laser pulse from the energy source. In still yet other embodiments, the station housing 260 can be manufactured utilizing another suitable method.

In many embodiments, the station housing 260 can have an integrally-formed, single-piece design, which can accommodate any suitable number of emitters 235. With the integrally-formed, single-piece design for the station housing 260, which includes and/or incorporates the housing base 262, the guide retainers 264, and the corresponding plasma targets 233, the same type of machining or manufacturing is required to produce the entire component, which, in some non-exclusive embodiments, can be machined from a tungsten rod. The advantage of this design is that the station housing 260 for each individual emitter station 280 can be produced as a standalone component. Thus, the overall design of the emitter system 231 and/or the individual emitter stations 280 can be manufactured and assembled in a much simpler, less time-consuming, and less expensive manner.

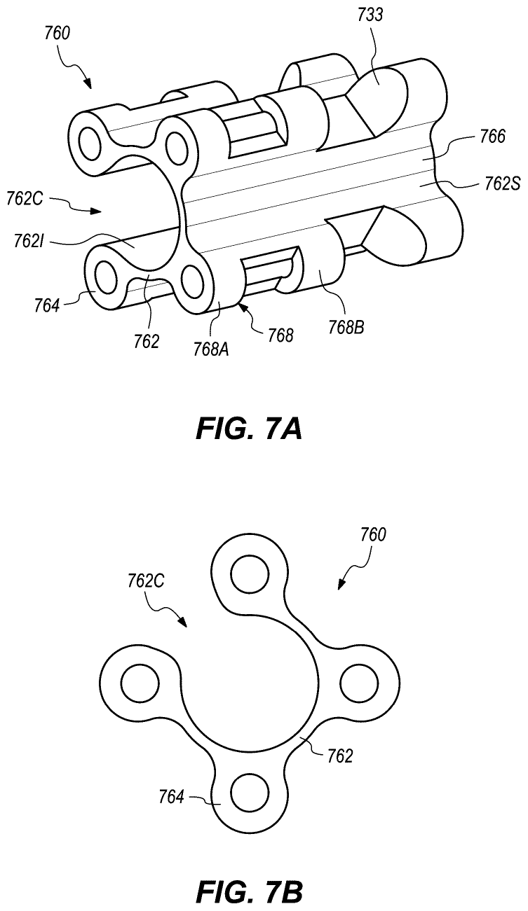

The housing base 262 of the station housing 260 can be substantially cylindrical-shaped, and can be configured to be threaded onto and/or positioned about the guidewire lumen 218. More particularly, in various embodiments, the housing base 262 and/or the station housing 260 can be mounted onto an end of the guidewire lumen 218, such as onto a distal end (not shown) of the guidewire lumen 218 or onto a proximal end (not shown) of the guidewire lumen 218, and then moved along the guidewire lumen 218 until the housing base 262 is positioned at the desired longitudinal position along the length of the guidewire lumen 218. Alternatively, in other embodiments of the emitter station 280A, 280B, the housing base 262 of the station housing 260 can be only partially cylindrical-shaped, with a base opening that enables the housing base 262 and/or the station housing 260 to be mounted about a side of the guidewire lumen 218 at any suitable or desired longitudinal position along the length of the guidewire lumen 218. With such alternative design, the housing base 262 and/or the station housing 260 would not need to be moved along the guidewire lumen 218 during the mounting process, but rather could simply be initially positioned at least partially about the guidewire lumen 218 at the desired longitudinal position along the length of the guidewire lumen 218. An example of such an embodiment of the emitter station 280, the station housing 260 and/or the housing base 262 is illustrated and described herein below in relation to FIGS. 7A and 7B.

In certain embodiments, an adhesive can be utilized to secure an inner (cylindrical) surface of the housing base 262 about an outer surface 218S of a substantially cylindrical-shaped and/or annular-shaped guidewire lumen 218. This design can thereby reduce the number of adhesive bands that were typically utilized and/or required with the previous design. More particularly, with the previous design, each energy guide for each emitter would be individually secured to the guidewire lumen, such as within a groove formed into the outer surface of the guidewire lumen, thus requiring four separate bonds for four energy guides (four emitters). Conversely, with the present design, only one bond is required to secure the station housing 260 and/or the housing base 262 about the guidewire lumen 218. This reduction from four bonds (for four emitters) to one bond (for four emitters), consequently reduces production time and potential points of failure. Moreover, with the present design, a heat shrink strap is no longer required to fixate the emitters of the emitter system within a grooved outer surface of the guidewire lumen.

As described in greater detail herein below, in certain embodiments, the housing base 262 can also include one or more channels, or grooves 266, formed into an outer surface 262S of the housing base 262 of the station housing 260. The grooves 266 can be used to help retain additional energy guides 222A that can effectively pass through a given emitter station 280, such as the first emitter station 280A, to be used as part of an emitter 235 at a subsequent emitter station 280, such as the second emitter station 280B. As described, the grooves 266 formed into the outer surface 262S of the housing base 262 help to position the additional energy guides 222A as the additional energy guides 222A extend past the housing base 262 at the first emitter station 280A and toward the housing base 262 at the second emitter station 280B. As such, the grooves 266 formed into the outer surface 262S of the housing base 262 can function as, or can sometimes be referred to as a “guide positioner”. It is appreciated that the station housing 260 and/or the housing base 262 can alternatively provide a guide positioner having a different design than what is specifically illustrated and described herein.

Thus, the present design further obviates the need for a grooved outer surface for the guidewire lumen to help manage positioning of the energy guides, as the energy guides 222A for the emitters 235 at subsequent emitter stations 280 can be effectively managed by securing the energy guides with a heat shrink band 270 in the grooves 266 that can be formed into the outer surface 262S of the housing base 262 of the station housing 260, such as shown in FIG. 2. Eliminating the need for the grooved outer surface for the guidewire lumen is an advantage because with current design, the energy guide must be bonded/fused to the guidewire lumen proximal to the emitter station. In the configuration illustrated and described in the present invention, a single guidewire extrusion can be used, therefore eliminating the need of the additional component and bond. Alternatively, the housing base 262 can be configured without grooves, and the positioning of the energy guides 222A can be maintained in another suitable manner as they pass through a given emitter station to be used at a subsequent emitter station.

As noted, the guide retainers 264 incorporated within the station housing 260, which extend away from the housing base 262, are each configured to selectively receive and retain the guide distal end 222D of one of the energy guides 222A, which are included as part of an individual emitter 235. In many embodiments, the guide retainers 264 of the station housing 260 are configured to be secured to and substantially encircle at least a portion of the energy guide 222A, such as at or near the guide distal end 222D of the energy guide 222A. As utilized herein, the description of each guide retainer 264 as substantially encircling at least a portion of one of the energy guides 222A is intended to signify that the guide retainer 264 encircles at least approximately 90% to 95% of such portion of the energy guide 222A, but can further include a small housing gap (not shown) that extends along a length of the guide retainer 264 and that allows for slight expansion or contraction of the guide retainer 264 due to changes in environmental conditions in which the catheter system 200 is being used.

A portion of the energy guide 222A, such as at or near the guide distal end 222D, can be secured within the guide retainer 264 in any suitable manner. For example, a portion of the energy guide 222A can be secured within the guide retainer 264 with any suitable type of adhesive material. Alternatively, a portion of the energy guide 222A can be secured within the guide retainer 264 in another suitable manner.

In the embodiment illustrated in FIG. 2, the guide retainers 264 are configured to extend radially outwardly away from the housing base 262. Alternatively, in another embodiment, the guide retainers 264 can extend radially inwardly away from the structure of the housing base 262. It is appreciated, however, that in such alternative embodiment, the housing base 262 will not generally have the same cylindrical inner surface for the housing base that is included in the embodiment illustrated in FIG. 2.

The guide retainers 264 can have any suitable design for purposes of effectively receiving and retaining the guide distal end 222D of individual energy guides 222A as part of the design of the individual emitters 235, as well as maintaining the desired positioning between the guide distal end 222D and the corresponding plasma target 233 for the individual emitter 235. As shown in FIG. 2, each guide retainer 264 can include two longitudinally spaced apart annular members 268, such as a first annular member 268A and a spaced apart second annular member 268B that are substantially longitudinally aligned with one another. As the guide distal end 222D of the energy guide 222A is being positioned to be received and retained by the guide retainer 264, the guide distal end 222D initially extends through the first annular member 268A before being received and retained within the second annular member 268B. The guide distal end 222D of each energy guide 222A can thus be properly aligned with the corresponding plasma target 233 for the individual emitter 235. Alternatively, the guide retainer 264 can have another suitable design for purposes of receiving and retaining the guide distal end 222D of an energy guide 222A, and maintaining the desired positioning between the guide distal end 222D and the corresponding plasma target 233 for the individual emitter 235. For example, in one non-exclusive alternative embodiment, each guide retainer 264 can be formed as a single annular member that extends away from the housing base 262.

As described herein, it is also appreciated that the corresponding plasma target 233 for each of the emitters 235 at the emitter station 280A, 280B are also integrally-formed with the guide retainers 264 and the housing base 262 as part of the station housing 260. With such design, when the guide distal end 222D of the energy guide 222A for each emitter 235 is secured as desired within the respective guide retainer 264, the desired spacing is necessarily provided between the guide distal end 222D of the energy guide 222A and the corresponding plasma target 233.

The plasma targets 233 that are thus included and/or incorporated as part of the station housing 260 can have any suitable design for purposes of generating plasma in the catheter fluid 232 within the balloon interior 246, and directing energy and/or pressure waves from the generated plasma toward the vascular lesions 106A at the treatment site 106. For example, in certain embodiments, the plasma target 233 can be provided in the form of a backstop-type structure with an angled face that redirects energy emitted from the guide distal end 222D toward the balloon wall 230 of the balloon 204 and/or toward the vessel wall 108A of the blood vessel 108 at the treatment site 106. Alternatively, the plasma target 233 can have another suitable structural design.

As noted above, the station housing 260, and thus the housing base 262, the guide retainers 264, and the corresponding plasma targets 233, can be formed from any suitable materials, such that the energy emitted from the guide distal end 222D of the energy guide 222A contacting the plasma target 233 generates the desired plasma in the catheter fluid 232 within the balloon interior 246. For example, in certain non-exclusive embodiments, the station housing 260, and thus the housing base 262, the guide retainers 264, and the corresponding plasma targets 233, can be formed, at least in part, from one or more metals and/or metal alloys having relatively high melting temperatures, such as titanium, stainless steel, tungsten, tantalum, platinum, molybdenum, niobium, iridium, etc. Alternatively, the station housing 260, and thus the housing base 262, the guide retainers 264, and the corresponding plasma targets 233, can be formed from at least one of magnesium oxide, beryllium oxide, tungsten carbide, titanium nitride, titanium carbonitride, and titanium carbide. Still alternatively, the station housing 260, and thus the housing base 262, the guide retainers 264, and the corresponding plasma targets 233, can be formed from at least one of chemical vapor deposition (CVD) diamond and diamond. In other embodiments, the station housing 260, and thus the housing base 262, the guide retainers 264, and the corresponding plasma targets 233, can be formed from a transition metal, an alloy metal, or a ceramic material. Yet alternatively, in some embodiments, the station housing 260, and thus the housing base 262, the guide retainers 264, and the corresponding plasma targets 233, can be formed at least partially from a polymer, a polymeric material, and/or a plastic such as polyimide and nylon. Still alternatively, the station housing 260, and thus the housing base 262, the guide retainers 264, and the corresponding plasma targets 233, can be formed from any other suitable materials.

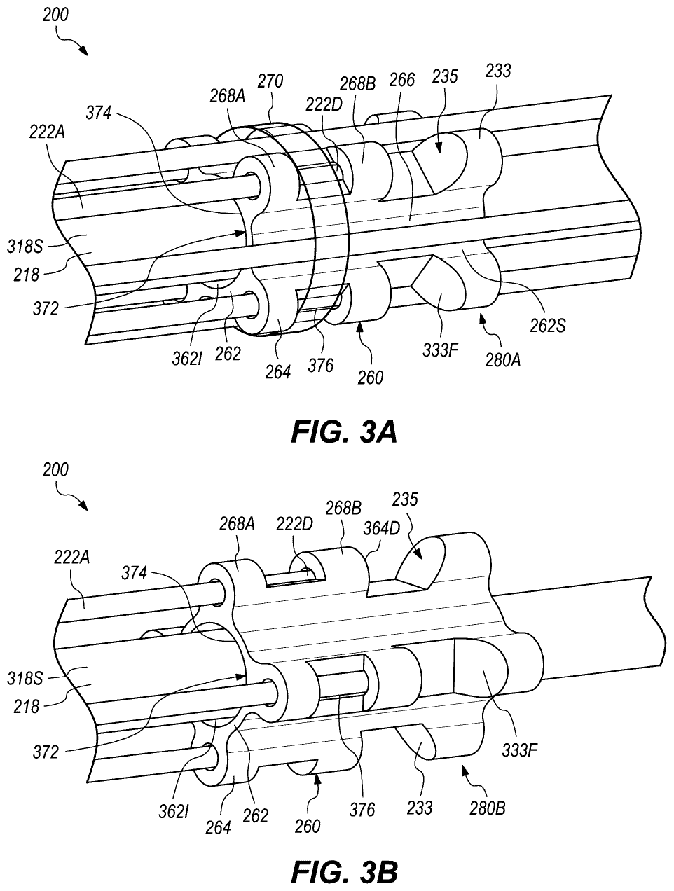

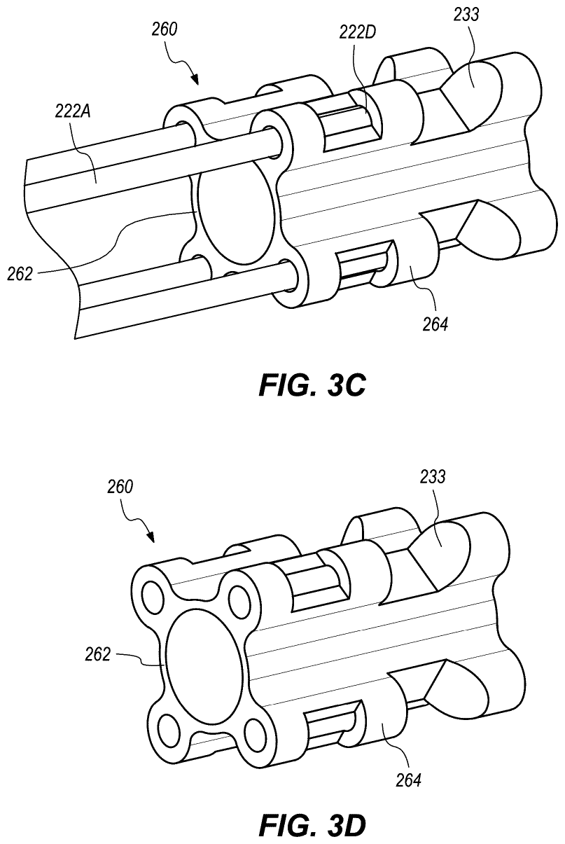

FIG. 3A is a simplified schematic perspective view illustration of a portion of the catheter system 200 illustrated in FIG. 2, including the first emitter station 280A. More particularly, FIG. 3A is a simplified schematic perspective view illustration showing a portion of the guidewire lumen 218, a portion of the energy guides 222A, and the first emitter station 280A.

As described herein, the first emitter station 280A includes an integrally-formed, single-piece station housing 260 that is configured to maintain the desired relative positioning and spacing between the guide distal end 222D of one or more energy guides 222A (the guide distal end 222D of four energy guides 222A are specifically incorporated into four individual emitters 235 at the first emitter station 280A in this particular embodiment) and one or more corresponding plasma targets 233 (four corresponding plasma targets 233 are specifically incorporated into the four individual emitters 235 at the first emitter station 280A in this particular embodiment). More specifically, in accordance with the teachings of the present invention, the integrally-formed, single-piece station housing 260 includes a housing base 262, one or more guide retainers 264 (four guide retainers 264 are included in this embodiment) that extend away from the housing base 262, and the corresponding plasma targets 233.

As shown in FIG. 3A, at the first emitter station 280A, the station housing 260 includes the housing base 262 that is generally cylindrical-shaped and/or annular-shaped and defines a generally cylindrical-shaped base aperture 372 that extends therethrough. As illustrated, the station housing 260 is positioned substantially about the guidewire lumen 218, such that the guidewire lumen 218 extends fully through the housing aperture 372. In many embodiments, as noted above, an adhesive 374 can be utilized to secure the station housing 260 to and/or about the guidewire lumen 218 at an appropriate and/or desired position along the length of the guidewire lumen 218. Alternatively, the station housing 260 can be secured to and/or about the guidewire lumen 218 in another suitable manner.

In this embodiment, the guidewire lumen 218 has a generally smooth, cylindrical-shaped outer surface 318S, and the housing base 262 has a corresponding, generally smooth, cylindrical-shaped inner surface 362I that defines the base aperture 372 that extends therethrough. With such design, the housing base 262 and/or the station housing 260 can be rotated about the guidewire lumen 218 such that the station housing 260 is secured in an appropriate and/or desired rotational position about the guidewire lumen 218, as well as being positioned at the appropriate and/or desired position along the length of the guidewire lumen 218. Additionally, or in the alternative, the outer surface 318S of the guidewire lumen 218 and/or the inner surface 362I of the housing base 262 can be threaded so that the housing base 262 can be threadedly mounted at a desired rotational position about the guidewire lumen 218.

The guide retainers 264 are integrally-formed with the remainder of the station housing 260 and, as noted, are configured to extend away from the housing base 262. In this particular embodiment, the guide retainers 264 are configured to extend radially outwardly away from the housing base 262.

Each guide retainer 264 is configured to receive and retain the guide distal end 222D of one of the energy guides 222A as part of an individual emitter 235. Since the first emitter station 280A, in this embodiment, is configured to include four individual emitters 235, the station housing 260 is configured to include four guide retainers 264 that extend radially outwardly away from the housing base 262. As shown in this embodiment, the four guide retainers 264 are substantially evenly spaced apart from one another, by approximately ninety degrees, radially about the housing base 262. Alternatively, the station housing 260 can include another suitable number of guide retainers 264, and/or the guide retainers 264 can be other than evenly spaced apart from one another.

As further shown in FIG. 3A, as described above, each guide retainer 264 can include a first annular member 268A and a corresponding, spaced apart, second annular member 268B. When the guide distal end 222D of one of the energy guides 222A is received and retained within the guide retainer 264, the guide distal end 222D first extends fully through the first annular member 268A before being effectively received and retained within the second annular member 268B. In certain implementations, the spacing between first annular member 268A and the second annular member 268B can function as a securing port 376 for receiving an adhesive to help securely retain the guide distal end 222D within the second annular member 268B of the guide retainer 264.

With the guide distal end 222D of the energy guide 222A being received and retained within the second annular member 268B, the guide distal end 222D is maintained a desired gap distance away from the corresponding plasma target 233. It is again noted that each of the corresponding plasma targets 233 (there are four corresponding plasma targets 233 in this embodiment) are also integrally formed with the remaining components of the station housing 260.

Thus, at the first emitter station 280A, four individual emitters 235 are provided, with each individual emitter 235 being configured such that energy from the energy source 124 (illustrated in FIG. 1) is emitted from the guide distal end 222D of the energy guide 222A toward the corresponding plasma target 233. The energy impinging on and/or contacting the plasma target 233 within each emitter 235 generates plasma in the catheter fluid 232 (illustrated in FIG. 2) within the balloon interior 246 (illustrated in FIG. 2). Energy from the generated plasma can then initiate pressure waves that are directed toward the balloon wall 230 (illustrated in FIG. 2) to break apart the vascular lesions 106A (illustrated in FIG. 1) at the treatment site 106 (illustrated in FIG. 1).

In some embodiments, the plasma target 233 can have an angled face 333F that helps in directing the energy from the plasma and/or the pressure waves as desired toward the balloon wall 230 to break apart the vascular lesions 106A at the treatment site 106. Thus, the angled face 333F acts like a single surface mirror. In some embodiments, the angled face 333F of the plasma target 233 can be angled at between approximately 5 degrees and 45 degrees relative to a flat, perpendicular configuration. Alternatively, the angled face 333F of the plasma target 233 can be angled at less than 5 degrees or greater than 45 degrees relative to a flat, perpendicular configuration in order to direct energy in the form of the plasma that has been generated in the catheter fluid 232 toward the balloon wall 230 positioned adjacent to the treatment site 106. Alternatively, the plasma target 233 can have another suitable structural design.

FIG. 3A also illustrates additional energy guides 222A that effectively pass through the first emitter station 280A and extend toward a subsequent emitter station, such as the second emitter station which is not shown in FIG. 3A. To maintain a desired positioning of the energy guides 222A as they pass through the first emitter station 280A, in certain embodiments, the housing base 262 can include one or more channels, or grooves 266, that are longitudinally formed into the outer surface 262S of the housing base 262 along a length of the housing base 262. Moreover, in some embodiments, a heat shrink band 270 can be positioned about the station housing 260 in order to more effectively maintain the desired positioning of the energy guides 222A relative to the station housing 260 as they pass through the first emitter station 280A.

It is appreciated that the integrally-formed, single-piece station housing 260 at the first emitter station 280A, with all of the components incorporated therein, can be formed from any suitable materials, such as noted above.

FIG. 3B is a simplified schematic perspective view illustration of another portion of the catheter system 200 illustrated in FIG. 2, including the second emitter station 280B. More particularly, FIG. 3B is a simplified schematic perspective view illustration showing a portion of the guidewire lumen 218, a portion of the energy guides 222A, and the second emitter station 280B.

As described herein, similar to the first emitter station 280A illustrated and described in relation to FIG. 3A, the second emitter station 280B also includes an integrally-formed, single-piece station housing 260 that is configured to maintain the desired relative positioning and spacing between the guide distal end 222D of one or more energy guides 222A (the guide distal end 222D of four energy guides 222A are specifically incorporated into four individual emitters 235 at the second emitter station 280B in this particular embodiment) and one or more corresponding plasma targets 233 (four corresponding plasma targets 233 are specifically incorporated into the four individual emitters 235 at the second emitter station 280B in this particular embodiment). More specifically, in accordance with the teachings of the present invention, the integrally-formed, single-piece station housing 260 again includes a housing base 262, one or more guide retainers 264 (four guide retainers 264 are included in this embodiment) that extend away from the housing base 262, and the corresponding plasma targets 233.

As shown in FIG. 3B, at the second emitter station 280B, the station housing 260 again includes the housing base 262 that is generally cylindrical-shaped and/or annular-shaped and defines a generally cylindrical-shaped base aperture 372 that extends therethrough. As illustrated, the station housing 260 is positioned substantially about the guidewire lumen 218, such that the guidewire lumen 218 extends fully through the housing aperture 372. In many embodiments, as noted above, an adhesive 374 can be utilized to secure the station housing 260 to and/or about the guidewire lumen 218 at an appropriate and/or desired position along the length of the guidewire lumen 218. Alternatively, the station housing 260 can be secured to and/or about the guidewire lumen 218 in another suitable manner.

In this embodiment, the guidewire lumen 218 again has a generally smooth, cylindrical-shaped outer surface 318S, and the housing base 262 has a corresponding, generally smooth, cylindrical-shaped inner surface 362I that defines the base aperture 372 that extends therethrough. With such design, the housing base 262 and/or the station housing 260 can be rotated about the guidewire lumen 218 such that the station housing 260 is secured in an appropriate and/or desired rotational position about the guidewire lumen 218, as well as being positioned at the appropriate and/or desired position along the length of the guidewire lumen 218. Additionally, or in the alternative, the outer surface 318S of the guidewire lumen 218 and/or the inner surface 362I of the housing base 262 can be threaded so that the housing base 262 can be threadedly mounted at a desired rotational position about the guidewire lumen 218.

The guide retainers 264 are integrally-formed with the remainder of the station housing 260 and, as noted, are configured to extend away from the housing base 262. In this particular embodiment, the guide retainers 264 are configured to extend radially outwardly away from the housing base 262.

Each guide retainer 264 is configured to receive and retain the guide distal end 222D of one of the energy guides 222A as part of an individual emitter 235. Since the second emitter station 280B, in this embodiment, is configured to include four individual emitters 235, the station housing 260 is configured to include four guide retainers 264 that extend radially outwardly away from the housing base 262. As shown in this embodiment, the four guide retainers 264 are substantially evenly spaced apart from one another, by approximately ninety degrees, radially about the housing base 262. Alternatively, the station housing 260 can include another suitable number of guide retainers 264, and/or the guide retainers 264 can be other than evenly spaced apart from one another.

As further shown in FIG. 3B, as described above, each guide retainer 264 can again include a first annular member 268A and a corresponding, spaced apart, second annular member 268B. When the guide distal end 222D of one of the energy guides 222A is received and retained within the guide retainer 264, the guide distal end 222D first extends fully through the first annular member 268A before being effectively received and retained within the second annular member 268B. In certain implementations, the spacing between first annular member 268A and the second annular member 268B can function as a securing port 376 for receiving an adhesive to help securely retain the guide distal end 222D within the second annular member 268B of the guide retainer 264.

With the guide distal end 222D of the energy guide 222A being received and retained within the second annular member 268B, the guide distal end 222D is maintained a desired gap distance away from the corresponding plasma target 233. More particularly, as shown, a member distal end 364D of the guide retainer 264 (which equates to a distal end of the second annular member 268B) is positioned spaced apart from the corresponding plasma target 233. With such design, when the guide distal end 222D of one of the energy guides 222A is positioned within the guide retainer 264 (within the second annular member 268B), the guide distal end 222D of the energy guide 222A will be spaced apart the desired gap distance away from the corresponding plasma target 233.

It is again noted that each of the corresponding plasma targets 233 (there are four corresponding plasma targets 233 in this embodiment) are also integrally formed with the remaining components of the station housing 260.

Thus, at the second emitter station 280B, four individual emitters 235 are provided, with each individual emitter 235 being configured such that energy from the energy source 124 (illustrated in FIG. 1) is emitted from the guide distal end 222D of the energy guide 222A toward the corresponding plasma target 233. The energy impinging on and/or contacting the plasma target 233 within each emitter 235 generates plasma in the catheter fluid 232 (illustrated in FIG. 2) within the balloon interior 246 (illustrated in FIG. 2). Energy from the generated plasma can then initiate pressure waves that are directed toward the balloon wall 230 (illustrated in FIG. 2) to break apart the vascular lesions 106A (illustrated in FIG. 1) at the treatment site 106 (illustrated in FIG. 1). In some embodiments, the plasma target 233 can have an angled face 333F that helps in directing the energy from the plasma and/or the pressure waves as desired toward the balloon wall 230 to break apart the vascular lesions 106A at the treatment site 106.

It is appreciated that the integrally-formed, single-piece station housing 260 at the second emitter station 280B, with all of the components incorporated therein, can again be formed from any suitable materials, such as noted above.

FIG. 3C is a simplified schematic perspective view illustration of an embodiment of the station housing 260 that can be included as part of one of the emitter stations 280A, 280B illustrated in FIG. 2, and a portion of the energy guides 222A that have been coupled to the station housing 260. In particular, because the guidewire lumen is no longer illustrated in FIG. 3C for purposes of clarity, certain features and aspects of the station housing 260 are more clearly visible.

As shown in FIG. 3C, the station housing 260 is again an integrally-formed, single-piece component that includes (i) the housing base 262, (ii) the guide retainers 264 that extend radially outwardly away from the housing base 262, and that are each configured to receive and retain the guide distal end 222D of one of the energy guides 222A, and (iii) the corresponding plasma targets 233. Various features and aspects of the integrally-formed, single-piece station housing 260 have been described in detail herein above. Accordingly, the station housing 260 will not again be described in detail in relation to FIG. 3C.

FIG. 3D is a simplified schematic perspective view illustration of the station housing 260 illustrated in FIG. 3C. In particular, because the energy guides are no longer illustrated in FIG. 3D for purposes of clarity, certain features and aspects of the station housing 260 are more clearly visible.

As again shown in FIG. 3D, the station housing 260 is an integrally-formed, single-piece component that includes (i) the housing base 262, (ii) the guide retainers 264 that extend radially outwardly away from the housing base 262, and that are each configured to receive and retain the guide distal end 222D (illustrated in FIG. 2) of one of the energy guides 222A (illustrated in FIG. 2), and (iii) the corresponding plasma targets 233. Various features and aspects of the integrally-formed, single-piece station housing 260 have been described in detail herein above. Accordingly, the station housing 260 will not again be described in detail in relation to FIG. 3D.

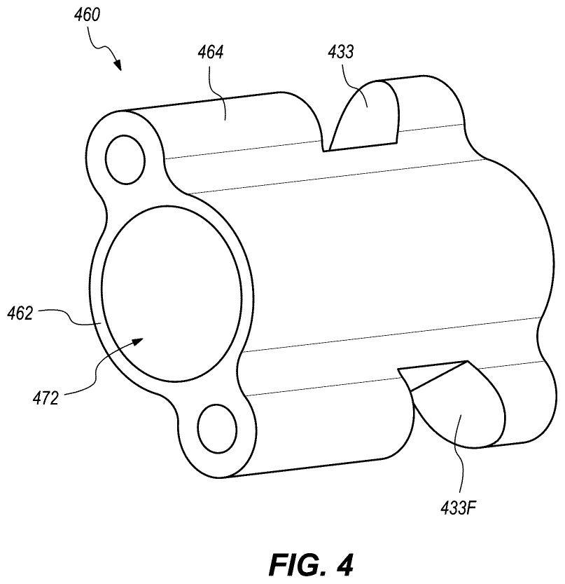

FIG. 4 is a simplified schematic perspective view illustration of another embodiment of the station housing 460 that can be included as part of one of the emitter stations 280A, 280B illustrated in FIG. 2. The station housing 460 is somewhat similar to the station housing 260 illustrated and described in detail herein above.

As shown in FIG. 4, the station housing 460 is again an integrally-formed, single-piece component that again includes (i) a housing base 462; (ii) one or more guide retainers 464 (two guide retainers 464 are included in this embodiment) that extend radially outwardly away from the housing base 462, and that are each configured to receive and retain the guide distal end 222D (illustrated in FIG. 2) of one of the energy guides 222A (illustrated in FIG. 2); and (iii) one or more corresponding plasma targets 433 (two corresponding plasma targets 433 are included in this embodiment).

In this embodiment, the housing base 462 is again generally cylindrical-shaped and/or annular-shaped and defines a generally cylindrical-shaped base aperture 472 that extends therethrough. When in use, the station housing 460 is positioned substantially about the guidewire lumen 218 (illustrated in FIG. 2), such that the guidewire lumen 218 extends fully through the housing aperture 472.

The guide retainers 464 are integrally-formed with the remainder of the station housing 460 and, in this embodiment, are configured to extend radially outwardly away from the housing base 262. As shown in this embodiment, the two guide retainers 464 are substantially evenly spaced apart from one another, by approximately 180 degrees, radially about the housing base 462. Alternatively, the guide retainers 464 can be other than evenly spaced apart from one another.

In this embodiment, each guide retainer 464 is substantially annular-shaped or tubular-shaped, and is configured to receive and retain the guide distal end 222D of one of the energy guides 222A. With the guide distal end 222D of the energy guide 222A being received and retained within the guide retainer 464, the guide distal end 222D is maintained a desired gap distance away from the corresponding plasma target 433. It is again noted that each of the corresponding plasma targets 433 (there are two corresponding plasma targets 433 in this embodiment) are also integrally formed with the remaining components of the station housing 460.

Thus, during use of this embodiment of the station housing 460, energy from the energy source 124 (illustrated in FIG. 1) is emitted from the guide distal end 222D (which is retained within one of the guide retainers 464) of the energy guide 222A toward the corresponding plasma target 433. The energy impinging on and/or contacting the plasma target 433 generates plasma in the catheter fluid 232 (illustrated in FIG. 2) within the balloon interior 246 (illustrated in FIG. 2). Energy from the generated plasma can then initiate pressure waves that are directed toward the balloon wall 230 (illustrated in FIG. 2) to break apart the vascular lesions 106A (illustrated in FIG. 1) at the treatment site 106 (illustrated in FIG. 1). In some embodiments, the plasma target 433 can have an angled face 433F that helps in directing the energy from the plasma and/or the pressure waves as desired toward the balloon wall 230 to break apart the vascular lesions 106A at the treatment site 106.

As with the previous embodiments, the station housing 460, and thus the housing base 462, the one or more guide retainers 464, and the one or more corresponding plasma targets 433, can be formed from any suitable materials, such that the energy emitted from the guide distal end 222D of the energy guide 222A contacting the plasma target 433 generates the desired plasma in the catheter fluid 232 within the balloon interior 246. For example, in certain non-exclusive embodiments, the station housing 460, and thus the housing base 462, the one or more guide retainers 464, and the one or more corresponding plasma targets 433, can be formed, at least in part, from one or more metals and/or metal alloys having relatively high melting temperatures, such as titanium, stainless steel, tungsten, tantalum, platinum, molybdenum, niobium, iridium, etc. Alternatively, the station housing 460, and thus the housing base 462, the one or more guide retainers 464, and the one or more corresponding plasma targets 433, can be formed from at least one of magnesium oxide, beryllium oxide, tungsten carbide, titanium nitride, titanium carbonitride, and titanium carbide. Still alternatively, the station housing 460, and thus the housing base 462, the one or more guide retainers 464, and the one or more corresponding plasma targets 433, can be formed from at least one of chemical vapor deposition (CVD) diamond and diamond. In other embodiments, the station housing 460, and thus the housing base 462, the one or more guide retainers 464, and the one or more corresponding plasma targets 433, can be formed from a transition metal, an alloy metal, or a ceramic material. Yet alternatively, in some embodiments, the station housing 460, and thus the housing base 462, the one or more guide retainers 464, and the one or more corresponding plasma targets 433, can be formed at least partially from a polymer, a polymeric material, and/or a plastic such as polyimide and nylon. Still alternatively, the station housing 460, and thus the housing base 462, the one or more guide retainers 464, and the one or more corresponding plasma targets 433, can be formed from any other suitable materials.

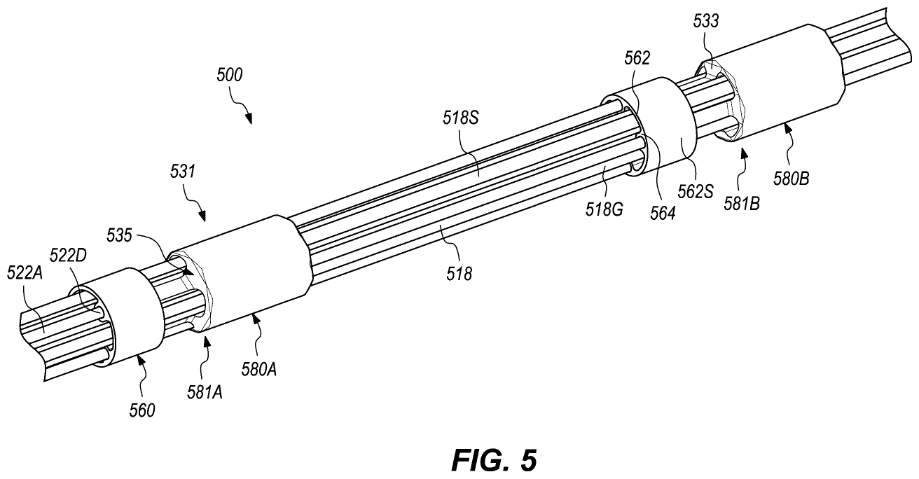

FIG. 5 is a simplified schematic perspective view illustration of a portion of another embodiment of the catheter system 500 illustrated in FIG. 1, including another embodiment of the emitter system 531 with a first emitter station 580A and a second emitter station 580B having features of the present invention. In particular, FIG. 5 is a simplified schematic perspective view illustration of a guidewire lumen 518, one or more energy guides 522A, and the emitter system 531, which can be included as part of the catheter system 500. The guidewire lumen 518, and the energy guides 522A are substantially similar in design and function to what was illustrated and described in detail herein above. Accordingly, such components will not be described in detail in relation to the embodiment illustrated in FIG. 5. It is appreciated that certain components of the catheter system 100 illustrated and described above in relation to FIG. 1, such as the balloon 104, the catheter shaft 110, the guidewire 112, the source manifold 136, the fluid pump 138, the handle assembly 129, the system console 123, the energy source 124, the power source 125, the system controller 126, the GUI 127, and the multiplexer 128, are not illustrated in FIG. 5 for purposes of simplicity and ease of illustration, but would typically be included in many embodiments.

The catheter system 500 can include any desired number of energy guides 522A. For example, in the embodiment illustrated in FIG. 5, the catheter system 500 includes eight energy guides 522A, with the guide distal end 522D of four of the energy guides 522A being received and retained at the first emitter station 580A, and the guide distal end 522D of another four energy guides 522A being received and retained at the second emitter station 580B. It is appreciated that not all eight energy guides 522A are visible in FIG. 5, as some energy guides 522A are hidden from view behind other components of the catheter system 500. Alternatively, the catheter system 500 can include greater than eight energy guides 522A or fewer than eight energy guides 522A, and/or a greater or lesser number of energy guides 522A can be received and retained at each of the emitter stations 580A, 580B.

In certain embodiments, the guidewire lumen 518 can be substantially annular-shaped and/or cylindrical-shaped and can have an outer surface 518S having one or more channels, or grooves 518G formed therein, with the grooves 518G extending in a generally longitudinal direction along the guidewire lumen 518. In such embodiments, each of the energy guides 522A and/or the emitter(s) 535 of the emitter system 531 can be positioned, received and retained within an individual groove 518G formed along and/or into the outer surface 518S of the guidewire lumen 518. More particularly, each of four energy guides 522A can be positioned, received and retained within an individual groove 518G as they extend toward and are received and retained, at least in part, at the first emitter station 580A; and each of the other four energy guides 522A can be further positioned, received and retained within an individual groove 518G as they extend through the first emitter station 580A and toward to be received and retained, at least in part, at the second emitter station 580B. Alternatively, the guidewire lumen 518 can be formed without a grooved outer surface 518S.

As noted above, in the embodiment shown in FIG. 5, the emitter system 531 includes the first emitter station 580A, which can be positioned at a first longitudinal position 581A relative to the length 242 (illustrated in FIG. 2) of the balloon 204 (illustrated in FIG. 2) and/or relative to the length of the guidewire lumen 518; and the second emitter station 580B, which can be positioned at a second longitudinal position 581B relative to the length 242 of the balloon 204 and/or relative to the length of the guidewire lumen 518. However, as with previous embodiments, it is further appreciated that the catheter system 500 can include any suitable number of emitter stations, which can alternatively be greater than two emitter stations, or only a single emitter station.

The first emitter station 580A can include any suitable number of emitters 535. More particularly, the first emitter station 580A can be configured to receive and retain the guide distal end 522D of any suitable number of energy guides 522A, which can each be included and/or incorporated into a corresponding emitter 535, and can further include any suitable number of corresponding plasma targets 533 that cooperate with the guide distal end 522D to form each individual emitter 535. In the embodiment illustrated in FIG. 5, the first emitter station 580A includes four individual emitters 535 that are radially spaced apart from one another by approximately ninety degrees. Stated in another manner, as noted above, the first emitter station 580A receives and retains the guide distal end 522D of four energy guides 522A, as well as incorporating four corresponding plasma targets 533, to form the four emitters 535 that are radially spaced apart from one another by approximately ninety degrees.

Similarly, the second emitter station 580B can include any suitable number of emitters 535. More particularly, the second emitter station 580B can be configured to receive and retain any suitable number of energy guides 522A, which can each include a guide distal end 522D that is included and/or incorporated into a corresponding emitter 535, and can further include any suitable number of plasma targets 533 that cooperate with the guide distal end 522D to form each individual emitter 535. In the embodiment illustrated in FIG. 5, the second emitter station 580B also includes four individual emitters 535 that are radially spaced apart from one another by approximately ninety degrees. Stated in another manner, as noted above, the second emitter station 580B receives and retains the guide distal end 522D of another four energy guides 522A, as well as incorporating four corresponding plasma targets 533, to form the four emitters 535 that are radially spaced apart from one another by approximately ninety degrees. As further illustrated in FIG. 5, the emitters 535 included at the second emitter station 580B are rotated approximately 45 degrees relative to the emitters 535 at the first emitter station 580A. The four energy guides 522A whose guide distal ends 522D form part of the four emitters 535 included at the second emitter station 580B are also shown as passing through the first emitter station 580A before extending on to the second emitter station 580B.

The design of the emitter stations 580A, 580B, and the individual emitters 535 within each emitter station 580A, 580B, can be varied to suit the requirements of the catheter system 500. In this embodiment, the design of each emitter station 580A, 580B can be substantially the same as one another. For example, in the embodiment illustrated in FIG. 5, each emitter station 580A, 580B includes a station housing 560 that is utilized to maintain a desired positioning between the guide distal end 522D of the energy guide 522A and the corresponding plasma target 533, for each of the emitters 535 included at the emitter station 580A, 580B. More particularly, since each emitter station 580A, 580B shown in FIG. 5 is configured to include four emitters 535, the station housing 560 is utilized to maintain a desired positioning between the guide distal end 522D of four energy guides 522A and the four corresponding plasma targets 533, for each of the four emitters 535 included at the emitter station 580A, 580B.

In several embodiments, the station housing 560 includes a housing base 562, one or more guide retainers 564 (four guide retainers 564 in this particular embodiment) that extend away from the housing base 562 and that are each configured to receive and retain at least the guide distal end 522D of one of the energy guides 522A, and one or more corresponding plasma targets 533 (four corresponding plasma targets 533 in this particular embodiment).

In many embodiments, the station housing 560 can have an integrally-formed, single-piece design, which can accommodate any suitable number of emitters 535. With the integrally-formed, single-piece design for the station housing 560, which includes and/or incorporates the housing base 562, the guide retainers 564, and the corresponding plasma targets 533, the same type of machining is required to produce the entire component, which, in some non-exclusive embodiments, can be machined from a tungsten rod. The advantage of this design is that the station housing 560 for each individual emitter station 580A, 580B can be produced as a standalone component. Thus, the overall design of the emitter system 531 and/or the individual emitter stations 580A, 580B can be manufactured and assembled in a much simpler, less time-consuming, and less expensive manner.

The housing base 562 of the station housing 560 can be somewhat cylindrical-shaped, and can be configured to be positioned on and/or about the guidewire lumen 518. In certain embodiments, an adhesive can be utilized to secure an inner surface of the housing base 562 about the outer surface 518S of a substantially cylindrical-shaped and/or annular-shaped guidewire lumen 518.

As noted, the guide retainers 564 incorporated within the station housing 560, which extend away from the housing base 562, are each configured to selectively receive and retain the guide distal end 522D of one of the energy guides 522A, which are included as part of an individual emitter 535. In the embodiment illustrated in FIG. 5, the guide retainers 564 are configured to extend radially inwardly away from the housing base 562.

It is appreciated that with the guide retainers 564 extending radially inwardly away from the housing base 562, and with the energy guides 522A often being retained within grooves 518G formed along the outer surface 518S of the guidewire lumen 518, there is no need to form grooves into an outer surface 562S of the housing base 562, such as were incorporated into certain previous embodiments.

The guide retainers 564 can have any suitable design for purposes of effectively receiving and retaining the guide distal end 522D of individual energy guides 522A as part of the design of the individual emitters 535, as well as maintaining the desired positioning between the guide distal end 522D and the corresponding plasma target 533 for the individual emitter 535. As shown in FIG. 5, each guide retainer 564 can be substantially annular-shaped or tubular-shaped, and can be configured to receive and retain the guide distal end 522D of one of the energy guides 522A. The guide distal end 522D of each of the energy guides 522A can be received and retained, and secured, within one of the guide retainers 564 in any suitable manner. For example, in certain non-exclusive embodiments, an adhesive material can be utilized to secure the guide distal end 522D of each energy guide 522A within one of the guide retainers 564. Alternatively, the guide distal end 522D of each energy guide 522A can be secured within one of the guide retainers 564 in another suitable manner.

With the guide distal end 522D of the energy guide 522A being received and retained within the guide retainer 564, the guide distal end 522D is maintained a desired gap distance away from the corresponding plasma target 533. More specifically, as described herein, it is also appreciated that the corresponding plasma target 533 for each of the emitters 535 at the emitter station 580A, 580B are also integrally-formed with the guide retainers 564 and the housing base 562 as part of the station housing 560. With such design, when the guide distal end 522D of the energy guide 522A for each emitter 535 is secured as desired within the respective guide retainer 564, the desired spacing is necessarily provided between the guide distal end 522D of the energy guide 522A and the corresponding plasma target 533.

The plasma targets 533 that are thus included and/or incorporated as part of the station housing 560 can have any suitable design for purposes of generating plasma in the catheter fluid 232 (illustrated in FIG. 2) within the balloon interior 246 (illustrated in FIG. 2), and directing energy and/or pressure waves from the generated plasma toward the vascular lesions 106A (illustrated in FIG. 1) at the treatment site 106 (illustrated in FIG. 1). For example, in certain embodiments, the plasma target 533 can be provided in the form of a backstop-type structure with an angled face that redirects energy emitted from the guide distal end 522D toward the balloon wall 230 (illustrated in FIG. 2) of the balloon 204 and/or toward the vessel wall 108A (illustrated in FIG. 1) of the blood vessel 108 (illustrated in FIG. 1) at the treatment site 106. Alternatively, the plasma target 533 can have another suitable structural design.

As with the previous embodiments, the station housing 560, and thus the housing base 562, the guide retainers 564, and the corresponding plasma targets 533, can be formed from any suitable materials, such that the energy emitted from the guide distal end 522D of the energy guide 522A contacting the plasma target 533 generates the desired plasma in the catheter fluid 232 within the balloon interior 246. For example, in certain non-exclusive embodiments, the station housing 560, and thus the housing base 562, the guide retainers 564, and the corresponding plasma targets 533, can be formed, at least in part, from one or more metals and/or metal alloys having relatively high melting temperatures, such as titanium, stainless steel, tungsten, tantalum, platinum, molybdenum, niobium, iridium, etc. Alternatively, the station housing 560, and thus the housing base 562, the guide retainers 564, and the corresponding plasma targets 533, can be formed from at least one of magnesium oxide, beryllium oxide, tungsten carbide, titanium nitride, titanium carbonitride, and titanium carbide. Still alternatively, the station housing 560, and thus the housing base 562, the guide retainers 564, and the corresponding plasma targets 533, can be formed from at least one of chemical vapor deposition (CVD) diamond and diamond. In other embodiments, the station housing 560, and thus the housing base 562, the guide retainers 564, and the corresponding plasma targets 533, can be formed from a transition metal, an alloy metal, or a ceramic material. Yet alternatively, in some embodiments, the station housing 560, and thus the housing base 562, the guide retainers 564, and the corresponding plasma targets 533, can be formed at least partially from a polymer, a polymeric material, and/or a plastic such as polyimide and nylon. Still alternatively, the station housing 560, and thus the housing base 562, the guide retainers 564, and the corresponding plasma targets 533, can be formed from any other suitable materials.

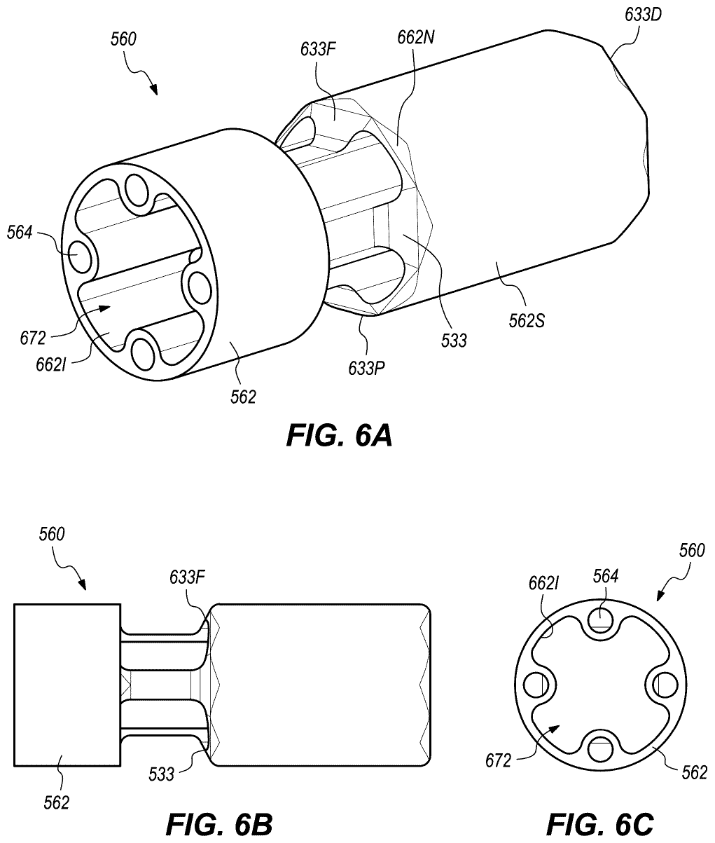

FIG. 6A is a simplified schematic perspective view illustration of an embodiment of the station housing 560 that can be included as part of one of the emitter stations 580A, 580B illustrated in FIG. 5. More particularly, FIG. 6A more clearly illustrates certain features and aspects of the station housing 560.

As described above, and as shown more clearly in FIG. 6A, the station housing 560 includes the housing base 562, the one or more guide retainers 564 (four guide retainers 564 are included in this particular embodiment) that extend radially inwardly away from an inner surface 662I of the housing base 562, and the one or more corresponding plasma targets 533 (four corresponding plasma targets 533 are included in this particular embodiment).

As shown in FIG. 6A, the housing base 562 has a generally smooth, cylindrical-shaped outer surface 562S. FIG. 6A further illustrates that the housing base 562 includes a base aperture 672 that extends therethrough. When the station housing 560 is positioned substantially about the guidewire lumen 518 (illustrated in FIG. 5), the inner surface 662I of the housing base 562 and/or the guide retainers 564 are configured to be positioned substantially directly adjacent to the outer surface 518S (illustrated in FIG. 5) of the guidewire lumen 518.

In some embodiments, the housing base 562 can include base notches 662N that are formed into the outer surface 562S of the housing base 562 substantially adjacent to the plasma targets 533, as well as between adjacent plasma targets 533. Stated in another manner, in such embodiments, the housing base 562 can include the base notches 562N circumferentially about the housing base 562 at or near a proximal end 633P of the plasma target 533 portion of the station housing 560. In certain embodiments, the housing base 562 can further include additional base notches 662N that are formed into the outer surface 562S of the housing base 562 at or near a distal end 633D of the plasma target 533 portion of the station housing 560. It is appreciated that any such base notches 662N can be provided to minimize any sharp edges that may otherwise be formed during the manufacturing of the station housing 560.

As noted above, and as shown in FIG. 6A, each guide retainer 564 can be substantially annular-shaped or tubular-shaped, and can be configured to receive and retain the guide distal end 522D (illustrated in FIG. 5) of one of the energy guides 522A (illustrated in FIG. 5). In this embodiment, a member distal end (not shown) of each guide retainer 564 is again positioned spaced apart from the corresponding plasma target 533. With such design, when the guide distal end 522D of one of the energy guides 522A is positioned within the guide retainer 564, the guide distal end 522D of the energy guide 522A will be spaced apart a desired gap distance away from the corresponding plasma target 533.

The guide distal end 522D of each of the energy guides 522A can be received and retained, and secured, within one of the guide retainers 564 in any suitable manner, such as with any suitable adhesive material in certain non-exclusive embodiments.

FIG. 6A also more clearly illustrates the design of the corresponding plasma targets 533. In particular, in this embodiment, each of the corresponding plasma targets 533 can be provided in the form of a backstop-type structure with an angled face 633F that redirects the energy emitted from the guide distal end 522D (illustrated in FIG. 5) of the energy guide 522A (illustrated in FIG. 5) in order to create the localized plasma and/or generate desired pressure waves in the catheter fluid 232 (illustrated in FIG. 2) within the balloon interior 246 (illustrated in FIG. 2) of the balloon 204 (illustrated in FIG. 2). Thus, the angled face 633F acts like a single surface mirror. In some embodiments, the angled face 633F of the plasma target 533 can be angled at between approximately 5 degrees and 45 degrees relative to a flat, perpendicular configuration. Alternatively, the angled face 633F of the plasma target 533 can be angled at less than 5 degrees or greater than 45 degrees relative to a flat, perpendicular configuration in order to direct energy in the form of the plasma that has been generated in the catheter fluid 232 toward the balloon wall 230 (illustrated in FIG. 2) positioned adjacent to the treatment site 106 (illustrated in FIG. 1). Still alternatively, the plasma target 533 can have another suitable design.

FIG. 6B is a simplified schematic side view illustration of the station housing 560 illustrated in FIG. 6A. More particularly, FIG. 6B provides an alternative view showing the housing base 562 and the plasma targets 533 incorporated within the station housing 560, as well as the angled face 633F of the plasma targets 533.

FIG. 6C is a simplified schematic end view illustration of the station housing 560 illustrated in FIG. 6A. More particularly, FIG. 6C provides another alternative view showing the housing base 562 and the guide retainers 564 that extend radially inwardly away from the inner surface 662I of the housing base 562. FIG. 6C also shows the base aperture 672 as defined by the housing base 562, for purposes of positioning the station housing 560 substantially about the guidewire lumen 518 (illustrated in FIG. 5).

FIG. 7A is a simplified schematic perspective view illustration of another embodiment of the station housing 760 that could be included as part of one of the emitter stations, such as one of the emitter stations 280A, 280B illustrated in FIG. 2.

More particularly, as illustrated, the station housing 760 is somewhat similar to the station housing 260 illustrated and described in detail in relation to FIG. 2. For example, as shown, the station housing 760 again includes a housing base 762, one or more guide retainers 764 (four guide retainers 764 in this particular embodiment) that extend away from the housing base 762 and that are each configured to receive and retain at least the guide distal end 222D (illustrated in FIG. 2) of one of the energy guides 222A (illustrated in FIG. 2), and one or more corresponding plasma targets 733 (four corresponding plasma targets 733 in this particular embodiment).

In the embodiment shown in FIG. 7A, the housing base 762 of the station housing 760 is only partially cylindrical-shaped, with a base opening 762C, or base channel, that enables the housing base 762 and/or the station housing 760 to be mounted about a side of the guidewire lumen 218 (illustrated in FIG. 2) at any suitable or desired longitudinal position along the length of the guidewire lumen 218. With such design, the housing base 762 and/or the station housing 760 would not need to be moved along the guidewire lumen 218 during the mounting process, but rather could simply be initially positioned at least partially about the guidewire lumen 218 at the desired longitudinal position along the length of the guidewire lumen 218.

As illustrated, the base opening 762C is sized such that it can be slightly expanded, or opened, as it fits about the guidewire lumen 218, but then moves back to its normal, relaxed size such that the housing base 762 and/or the station housing 760 fits snugly about the guidewire lumen 218. In some embodiments, the base opening 762C can be sized such that the housing base 762 is between approximately 10% and 40% less than fully cylindrical-shaped. More particularly, in certain non-exclusive alternative embodiments, the base opening 762C can be sized such that the housing base 762 is approximately 10%, 15%, 20%, 25%, 30%, 35%, or 40% less than fully cylindrical-shaped. Alternatively, the base opening 762C can have another suitable size for purposes of being able to be mounted about a side of the guidewire lumen 218, while still being able to be effectively maintained in the desired longitudinal position along the length of the guidewire lumen 218.

It is appreciated that the snug fitting of the housing base 762 and/or the station housing 760 about the guidewire lumen 218 can provide a frictional force that helps to maintain the station housing 760 and/or the housing base 762 in the desired longitudinal position relative to the length of the guidewire lumen 218. However, in certain embodiments, an adhesive can also be utilized to secure an inner surface 762I of the housing base 762 about the outer surface 218S (illustrated in FIG. 2) of the substantially cylindrical-shaped and/or annular-shaped guidewire lumen 218. This design can again reduce the number of adhesive bands that were typically utilized and/or required with the previous design, in which each energy guide for each emitter would be individually secured to the guidewire lumen 218.

As further illustrated, in certain embodiments, the housing base 762 can again include one or more channels, or grooves 766, formed into an outer surface 762S of the housing base 762 of the station housing 760. The grooves 766 can again be used to help retain additional energy guides 222A that can effectively pass through a given emitter station 280 (illustrated in FIG. 2), such as the first emitter station 280A (illustrated in FIG. 2), to be used as part of an emitter 235 (illustrated in FIG. 2) at a subsequent emitter station 280, such as the second emitter station 280B (illustrated in FIG. 2). As described, the grooves 766 formed into the outer surface 762S of the housing base 762 help to position the additional energy guides 222A as the additional energy guides 222A extend past the housing base 762 at the first emitter station 280A and toward the housing base 762 at the second emitter station 280B. Thus, this design also further obviates the need for a grooved outer surface for the guidewire lumen 218 to help manage positioning of the energy guides 222A.

As noted, the guide retainers 764 incorporated within the station housing 760, which extend away from the housing base 762, are again each configured to selectively receive and retain the guide distal end 222D of one of the energy guides 222A, which are included as part of an individual emitter 235. In many embodiments, the guide retainers 764 of the station housing 760 are again configured to be secured to and substantially encircle at least a portion of the energy guide 222A, such as at or near the guide distal end 222D of the energy guide 222A.

A portion of the energy guide 222A, such as at or near the guide distal end 222D, can be secured within the guide retainer 764 in any suitable manner. For example, a portion of the energy guide 222A can be secured within the guide retainer 764 with any suitable type of adhesive material. Alternatively, a portion of the energy guide 222A can be secured within the guide retainer 764 in another suitable manner.

In the embodiment illustrated in FIG. 7A, the guide retainers 764 are again configured to extend radially outwardly away from the housing base 762. Alternatively, the guide retainers 764 can extend radially inwardly away from the structure of the housing base 762.

The guide retainers 764 can have any suitable design for purposes of effectively receiving and retaining the guide distal end 222D of individual energy guides 222A as part of the design of the individual emitters 235, as well as maintaining the desired positioning between the guide distal end 222D and the corresponding plasma target 733 for the individual emitter 235. As shown in FIG. 7A, each guide retainer 764 can again include two longitudinally spaced apart annular members 768, such as a first annular member 768A and a spaced apart second annular member 768B that are substantially longitudinally aligned with one another. As the guide distal end 222D of the energy guide 222A is being positioned to be received and retained by the guide retainer 764, the guide distal end 222D initially extends through the first annular member 768A before being received and retained within the second annular member 768B. The guide distal end 222D of each energy guide 222A can thus be properly aligned with the corresponding plasma target 733 for the individual emitter 235. Alternatively, the guide retainer 764 can have another suitable design for purposes of receiving and retaining the guide distal end 222D of an energy guide 222A, and maintaining the desired positioning between the guide distal end 222D and the corresponding plasma target 733 for the individual emitter 235.

As described herein, it is also appreciated that the corresponding plasma target 733 for each of the emitters 235 at the emitter station 280A, 280B are also integrally-formed with the guide retainers 764 and the housing base 762 as part of the station housing 760. With such design, when the guide distal end 222D of the energy guide 222A for each emitter 235 is secured as desired within the respective guide retainer 764, the desired spacing is necessarily provided between the guide distal end 222D of the energy guide 222A and the corresponding plasma target 733.

The plasma targets 733 that are thus included and/or incorporated as part of the station housing 760 can have any suitable design for purposes of generating plasma in the catheter fluid 232 (illustrated in FIG. 2) within the balloon interior 246 (illustrated in FIG. 2), and directing energy and/or pressure waves from the generated plasma toward the vascular lesions 106A (illustrated in FIG. 1) at the treatment site 106 (illustrated in FIG. 1). For example, in certain embodiments, the plasma target 733 can again be provided in the form of a backstop-type structure with an angled face that redirects energy emitted from the guide distal end 222D toward the balloon wall 230 (illustrated in FIG. 2) of the balloon 204 (illustrated in FIG. 2) and/or toward the vessel wall 108A (illustrated in FIG. 1) of the blood vessel 108 (illustrated in FIG. 1) at the treatment site 106. Alternatively, the plasma target 733 can have another suitable structural design.

FIG. 7B is a simplified schematic end view illustration of the station housing 760 illustrated in FIG. 7A. More particularly, FIG. 7B illustrates a different view of at least the housing base 762, including the base opening 762C, and the guide retainers 764 of the station housing 760.

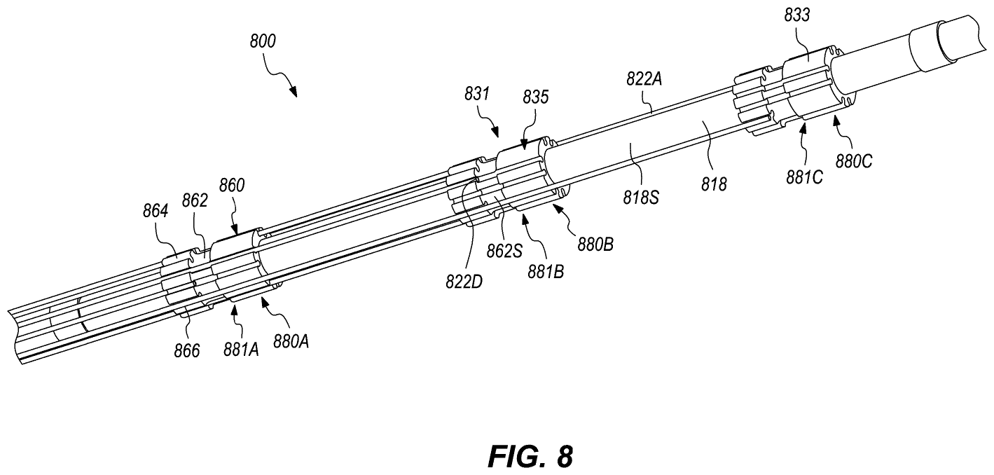

FIG. 8 is a simplified schematic perspective view illustration of a portion of still another embodiment of the catheter system 800, including still another embodiment of the emitter system 831 with a first emitter station 880A, a second emitter station 880B and a third emitter station 880C having features of the present invention. In particular, FIG. 8 is a simplified schematic perspective view illustration of a guidewire lumen 818, one or more energy guides 822A, and the emitter system 831, which can be included as part of the catheter system 800. The guidewire lumen 818, and the energy guides 822A are substantially similar in design and function to what was illustrated and described in detail herein above. Accordingly, such components will not be described in detail in relation to the embodiment illustrated in FIG. 8. It is appreciated that certain components of the catheter system 100 illustrated and described above in relation to FIG. 1, such as the balloon 104, the catheter shaft 110, the guidewire 112, the source manifold 136, the fluid pump 138, the handle assembly 129, the system console 123, the energy source 124, the power source 125, the system controller 126, the GUI 127, and the multiplexer 128, are not illustrated in FIG. 8 for purposes of simplicity and ease of illustration, but would typically be included in many embodiments.

The catheter system 800 can include any desired number of energy guides 822A. For example, in the embodiment illustrated in FIG. 8, the catheter system 800 includes twelve energy guides 822A, with the guide distal end 822D of four of the energy guides 822A being received and retained at each of the first emitter station 880A, the second emitter station 880B, and the third emitter station 880C. It is appreciated that not all twelve energy guides 822A are visible in FIG. 8, as some energy guides 822A are hidden from view behind other components of the catheter system 800. Alternatively, the catheter system 800 can include greater than twelve energy guides 822A or fewer than twelve energy guides 822A, and/or a greater or lesser number of energy guides 822A can be received and retained at each of the emitter stations 880A, 880B, 880C.

As noted above, in the embodiment shown in FIG. 8, the emitter system 831 includes the first emitter station 880A, which can be positioned at a first longitudinal position 881A relative to the length 242 (illustrated in FIG. 2) of the balloon 204 (illustrated in FIG. 2) and/or relative to the length of the guidewire lumen 818; the second emitter station 880B, which can be positioned at a second longitudinal position 881B relative to the length 242 of the balloon 204 and/or relative to the length of the guidewire lumen 818; and the third emitter station 880C, which can be positioned at a third longitudinal position 881C relative to the length 242 of the balloon 204 and/or relative to the length of the guidewire lumen 818. However, as with previous embodiments, it is further appreciated that the catheter system 800 can include any suitable number of emitter stations, which can alternatively be greater than or less than three emitter stations.

The first emitter station 880A can include any suitable number of emitters 835. More particularly, the first emitter station 880A can be configured to receive and retain the guide distal end 822D of any suitable number of energy guides 822A, which can each be included and/or incorporated into a corresponding emitter 835, and can further include any suitable number of corresponding plasma targets 833 that cooperate with the guide distal end 822D to form each individual emitter 835. In the embodiment illustrated in FIG. 8, the first emitter station 880A includes four individual emitters 835 that are radially spaced apart from one another by approximately ninety degrees. Stated in another manner, as noted above, the first emitter station 880A receives and retains the guide distal end 822D of four energy guides 822A, as well as incorporating four corresponding plasma targets 833, to form the four emitters 835 that are radially spaced apart from one another by approximately ninety degrees.

Similarly, the second emitter station 880B can include any suitable number of emitters 835. More particularly, the second emitter station 880B can be configured to receive and retain any suitable number of energy guides 822A, which can each include a guide distal end 822D that is included and/or incorporated into a corresponding emitter 835, and can further include any suitable number of plasma targets 833 that cooperate with the guide distal end 822D to form each individual emitter 835. In the embodiment illustrated in FIG. 8, the second emitter station 880B also includes four individual emitters 835 that are radially spaced apart from one another by approximately ninety degrees. Stated in another manner, as noted above, the second emitter station 880B receives and retains the guide distal end 822D of another four energy guides 822A, as well as incorporating four corresponding plasma targets 833, to form the four emitters 835 that are radially spaced apart from one another by approximately ninety degrees. As further illustrated in FIG. 8, the emitters 835 included at the second emitter station 580B are rotated approximately 30 degrees relative to the emitters 835 at the first emitter station 880A. The four energy guides 822A whose guide distal ends 822D form part of the four emitters 835 included at the second emitter station 880B are also shown as passing through the first emitter station 880A before extending on to the second emitter station 880B.

Still similarly, the third emitter station 880C can also include any suitable number of emitters 835. More particularly, the third emitter station 880C can also be configured to receive and retain any suitable number of energy guides 822A, which can each include a guide distal end 822D that is included and/or incorporated into a corresponding emitter 835, and can further include any suitable number of plasma targets 833 that cooperate with the guide distal end 822D to form each individual emitter 835. In the embodiment illustrated in FIG. 8, the third emitter station 880C also includes four individual emitters 835 that are radially spaced apart from one another by approximately ninety degrees. Stated in another manner, as noted above, the third emitter station 880C receives and retains the guide distal end 822D of still another four energy guides 822A, as well as incorporating four corresponding plasma targets 833, to form the four emitters 835 that are radially spaced apart from one another by approximately ninety degrees. As further illustrated in FIG. 8, the emitters 835 included at the third emitter station 880C are rotated approximately 30 degrees relative to the emitters 835 at the first emitter station 880A, and are also rotated approximately 30 degrees relative to the emitters 835 at the second emitter station 880B. The four energy guides 822A whose guide distal ends 822D form part of the four emitters 835 included at the third emitter station 880C are also shown as passing through the first emitter station 880A and the second emitter station 880B before extending on to the third emitter station 880C.

The design of the emitter stations 880A, 880B, 880C, and the individual emitters 835 within each emitter station 880A, 880B, 880C, can be varied to suit the requirements of the catheter system 800. In this embodiment, the design of each emitter station 880A, 880B, 880C can be substantially the same as one another. For example, in the embodiment illustrated in FIG. 8, each emitter station 880A, 880B, 880C includes a station housing 860 that is utilized to maintain a desired positioning between the guide distal end 822D of the energy guide 822A and the corresponding plasma target 833, for each of the emitters 835 included at the emitter station 880A, 880B, 880C. More particularly, since each emitter station 880A, 880B, 880C shown in FIG. 8 is configured to include four emitters 835, the station housing 860 is utilized to maintain a desired positioning between the guide distal end 822D of four energy guides 822A and the four corresponding plasma targets 833, for each of the four emitters 835 included at the emitter station 880A, 880B, 880C.

In several embodiments, the station housing 860 includes a housing base 862, one or more guide retainers 864 (four guide retainers 864 in this particular embodiment) that extend away from the housing base 862 and that are each configured to receive and retain at least the guide distal end 822D of one of the energy guides 822A, and one or more corresponding plasma targets 833 (four corresponding plasma targets 833 in this particular embodiment).

As with the previous embodiments, the station housing 860 can have an integrally-formed, single-piece design, which can accommodate any suitable number of emitters 835. With the integrally-formed, single-piece design for the station housing 860, which includes and/or incorporates the housing base 862, the guide retainers 864, and the corresponding plasma targets 833, the same type of machining is required to produce the entire component, which, as noted above with regard to previous embodiments, can be machined from a tungsten rod, or can be manufactured in another suitable manner. As before, the advantage of this design is that the station housing 860 for each individual emitter station 880A, 880B, 880C can be produced as a standalone component. Thus, the overall design of the emitter system 831 and/or the individual emitter stations 880A, 880B, 880C can be manufactured and assembled in a much simpler, less time-consuming, and less expensive manner.

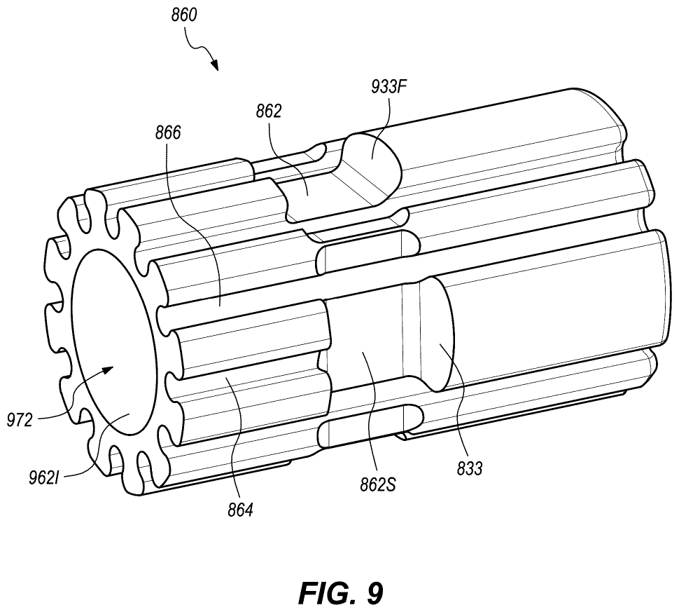

The housing base 862 of the station housing 860 can be substantially cylindrical-shaped and/or annular-shaped, and can be configured to be positioned on and/or about the guidewire lumen 818. In certain embodiments, an adhesive can be utilized to secure an inner surface 962I (illustrated in FIG. 9) of the housing base 862 about the outer surface 818S of a substantially cylindrical-shaped and/or annular-shaped guidewire lumen 818.

As noted, the guide retainers 864 incorporated within the station housing 860, which extend away from the housing base 862, are each configured to selectively receive and retain the guide distal end 822D of one of the energy guides 822A, which are included as part of an individual emitter 835. In the embodiment illustrated in FIG. 8, the guide retainers 864 are configured to extend radially outwardly away from the housing base 862. More specifically, as shown in this embodiment, the guide retainers 864 are provided in the form of grooves that are created outwardly away from the housing base 862 and/or are formed into an outer surface 862S of the housing base 862. As such, the guide retainers 864 can effectively receive and retain the guide distal end 822D of individual energy guides 822A, while also maintaining the desired positioning between the guide distal end 822D and the corresponding plasma target 833 for the individual emitter 835. The guide distal end 822D of each of the energy guides 822A can be received and retained, and secured, within one of the guide retainers 864 in any suitable manner. For example, in certain non-exclusive embodiments, an adhesive material can be utilized to secure the guide distal end 822D of each energy guide 822A within one of the guide retainers 864. Additionally, and/or alternatively, a heat-shrink style attacher can be utilized to secure the guide distal end 822D of each energy guide 822A within one of the guide retainers 864. Still alternatively, the guide distal end 822D of each energy guide 822A can be secured within one of the guide retainers 864 in another suitable manner.

With the guide distal end 822D of the energy guide 822A being received and retained within the guide retainer 864, the guide distal end 822D is maintained a desired gap distance away from the corresponding plasma target 833. More specifically, as described herein, it is also appreciated that the corresponding plasma target 833 for each of the emitters 835 at the emitter station 880A, 880B, 880C are also integrally-formed with the guide retainers 864 and the housing base 862 as part of the station housing 860. With such design, when the guide distal end 822D of the energy guide 822A for each emitter 835 is secured as desired within the respective guide retainer 864, the desired spacing is necessarily provided between the guide distal end 822D of the energy guide 822A and the corresponding plasma target 833.

The plasma targets 833 that are thus included and/or incorporated as part of the station housing 860 can have any suitable design for purposes of generating plasma in the catheter fluid 232 (illustrated in FIG. 2) within the balloon interior 246 (illustrated in FIG. 2), and directing energy and/or pressure waves from the generated plasma toward the vascular lesions 106A (illustrated in FIG. 1) at the treatment site 106 (illustrated in FIG. 1). For example, in certain embodiments, the plasma target 833 can again be provided in the form of a backstop-type structure with an angled face that redirects energy emitted from the guide distal end 822D toward the balloon wall 230 (illustrated in FIG. 2) of the balloon 204 (illustrated in FIG. 2) and/or toward the vessel wall 108A (illustrated in FIG. 1) of the blood vessel 108 (illustrated in FIG. 1) at the treatment site 106. Alternatively, the plasma target 833 can have another suitable structural design.

As further illustrated, the station housing 860 further includes additional grooves 866 that are formed into the outer surface 862S of the housing base 862 that are substantially similar to the grooves formed into the outer surface 862S of the housing base 862 that function as the guide retainers 564 extending radially outwardly away from the housing base 562. However, these additional grooves 866 do not have a corresponding plasma target, but rather are configured to receive and retain a portion of the energy guides 822A that pass through the particular emitter station 880A, 880B before extending on to a subsequent emitter station 880B, 880C. As such, the position of the energy guides 822A as they pass through a given emitter station 880A, 880B before extending on to a subsequent emitter station 880B, 880C can be effectively maintained. It is appreciated that an adhesive material and/or a heat-shrink style attacher can further be utilized to help maintain the position of the energy guides 822A as they pass through a given emitter station 880A, 880B before extending on to a subsequent emitter station 880B, 880C.

As with the previous embodiments, the station housing 860, and thus the housing base 862, the guide retainers 864, and the corresponding plasma targets 833, as well as the additional grooves 866, can be formed from any suitable materials, such that the energy emitted from the guide distal end 822D of the energy guide 822A contacting the plasma target 833 generates the desired plasma in the catheter fluid 232 within the balloon interior 246. For example, in certain non-exclusive embodiments, the station housing 860, and thus the housing base 862, the guide retainers 864, and the corresponding plasma targets 833, as well as the additional grooves 866, can be formed, at least in part, from one or more metals and/or metal alloys having relatively high melting temperatures, such as titanium, stainless steel, tungsten, tantalum, platinum, molybdenum, niobium, iridium, etc. Alternatively, the station housing 860, and thus the housing base 862, the guide retainers 864, and the corresponding plasma targets 833, as well as the additional grooves 866, can be formed from at least one of magnesium oxide, beryllium oxide, tungsten carbide, titanium nitride, titanium carbonitride, and titanium carbide. Still alternatively, the station housing 860, and thus the housing base 862, the guide retainers 864, and the corresponding plasma targets 833, as well as the additional grooves 866, can be formed from at least one of chemical vapor deposition (CVD) diamond and diamond. In other embodiments, the station housing 860, and thus the housing base 862, the guide retainers 864, and the corresponding plasma targets 833, as well as the additional grooves 866, can be formed from a transition metal, an alloy metal, or a ceramic material. Yet alternatively, in some embodiments, the station housing 860, and thus the housing base 862, the guide retainers 864, and the corresponding plasma targets 833, as well as the additional grooves 866, can be formed at least partially from a polymer, a polymeric material, and/or a plastic such as polyimide and nylon. Still alternatively, the station housing 860, and thus the housing base 862, the guide retainers 864, and the corresponding plasma targets 833, as well as the additional grooves 866, can be formed from any other suitable materials.

FIG. 9 is a simplified schematic perspective view illustration of an embodiment of the station housing 860 that can be included as part of one of the emitter stations 880A, 880B, 880C illustrated in FIG. 8. More particularly, FIG. 9 more clearly illustrates certain features and aspects of the station housing 860.

As described above, and as shown more clearly in FIG. 9, the station housing 860 includes the housing base 862, the one or more guide retainers 864 (four guide retainers 864 are included in this particular embodiment) that extend radially outwardly away from an outer surface 862S of the housing base 862, the one or more corresponding plasma targets 833 (four corresponding plasma targets 833 are included in this particular embodiment), and the additional grooves 866 that are formed into the outer surface 862S of the housing base 862 that are substantially similar to the grooves formed into the outer surface 862S of the housing base 862 that function as the guide retainers 864 extending radially outwardly away from the housing base 862.

As shown in FIG. 9, the housing base 862 has a generally smooth, cylindrical-shaped inner surface 962I that is configured to be positioned at least substantially if not fully about the guidewire lumen 818 (illustrated in FIG. 8) during use of the catheter system 800 (illustrated in FIG. 8). FIG. 9 further illustrates that the housing base 862 includes a base aperture 972 that extends therethrough. When the station housing 860 is positioned substantially about the guidewire lumen 818, the inner surface 962I of the housing base 862 is configured to be positioned substantially directly adjacent to the outer surface 818S (illustrated in FIG. 8) of the guidewire lumen 818.

As noted above, and as illustrated in the embodiment shown in FIG. 9, each guide retainer 864 can be provided in the form of a groove that is created outwardly away from the housing base 862 and/or is formed into the outer surface 862S of the housing base 862. As such, the guide retainers 864 can effectively receive and retain the guide distal end 822D (illustrated in FIG. 8) of individual energy guides 822A (illustrated in FIG. 8), while also maintaining the desired positioning between the guide distal end 822D and the corresponding plasma target 833.

As further noted above, it is appreciated that the guide distal end 822D of each of the energy guides 822A can be received and retained, and secured, within one of the guide retainers 864 in any suitable manner, such as with any suitable adhesive material and/or a heat-shrink style attacher in certain non-exclusive embodiments.

FIG. 9 also more clearly illustrates the design of the corresponding plasma targets 833. In particular, in this embodiment, each of the corresponding plasma targets 833 can be provided in the form of a backstop-type structure with an angled face 933F that redirects the energy emitted from the guide distal end 822D of the energy guide 822A in order to create the localized plasma and/or generate desired pressure waves in the catheter fluid 232 (illustrated in FIG. 2) within the balloon interior 246 (illustrated in FIG. 2) of the balloon 204 (illustrated in FIG. 2). Thus, the angled face 833F acts like a single surface mirror. In some embodiments, the angled face 833F of the plasma target 833 can be angled at between approximately 5 degrees and 45 degrees relative to a flat, perpendicular configuration. Alternatively, the angled face 833F of the plasma target 833 can be angled at less than 5 degrees or greater than 45 degrees relative to a flat, perpendicular configuration in order to direct energy in the form of the plasma that has been generated in the catheter fluid 232 toward the balloon wall 230 (illustrated in FIG. 2) positioned adjacent to the treatment site 106 (illustrated in FIG. 1). Still alternatively, the plasma target 833 can have another suitable design.

As further shown in this embodiment, the corresponding plasma target 833, such as in the form of a backstop-type structure with an angled face 933F, can be somewhat larger than is possible in other embodiments. More particularly, because this embodiment includes guide retainers 864 that are provided in the form of grooves, rather than as substantially full cylindrical-shaped and/or annular-shaped guide retainers as in other embodiments, there is more spacing to enable the angled face 933F to be somewhat larger than is possible in other embodiments. It is appreciated that the larger size for the angled face 933F and/or the corresponding plasma target 833 can have a potential to positively impact the acoustic output of the catheter system 800.

FIG. 9 also more clearly illustrates the additional grooves 866 that can be formed into the outer surface 862S of the housing base 862. As noted above, these additional grooves 866 do not have a corresponding plasma target, but rather are configured to receive and retain a portion of the energy guides 822A that pass through the particular emitter station 880A, 880B (illustrated in FIG. 8) before extending on to a subsequent emitter station 880B, 880C (illustrated in FIG. 8). As such, the position of the energy guides 822A as they pass through a given emitter station 880A, 880B before extending on to a subsequent emitter station 880B, 880C can be effectively maintained. It is again appreciated that an adhesive material and/or a heat-shrink style attacher can further be utilized to help maintain the position of the energy guides 822A as they pass through a given emitter station 880A, 880B before extending on to a subsequent emitter station 880B, 880C.

With this particular design for the station housing 860, it is appreciated that the catheter 102 (illustrated in FIG. 1) can be assembled in a top-down type manner. For example, the energy guides 822A can be added after the emitter stations 880A, 880B, 880C have already been attached to the guidewire lumen 818.

Many embodiments of the emitter station have been illustrated and described herein, which include a station housing that is integrally formed as a unitary, single-piece component that is configured to maintain a desired positioning between the guide distal end of the energy guide and the corresponding plasma target for each of the emitters included at the emitter station. In any such embodiments, the station housing can further incorporate a distal edge, or leading edge, that is slanted (angled other than perpendicular) to help the station housing lead into the one or more lesions 106A (illustrated in FIG. 1) at the treatment site 106 (illustrated in FIG. 1).

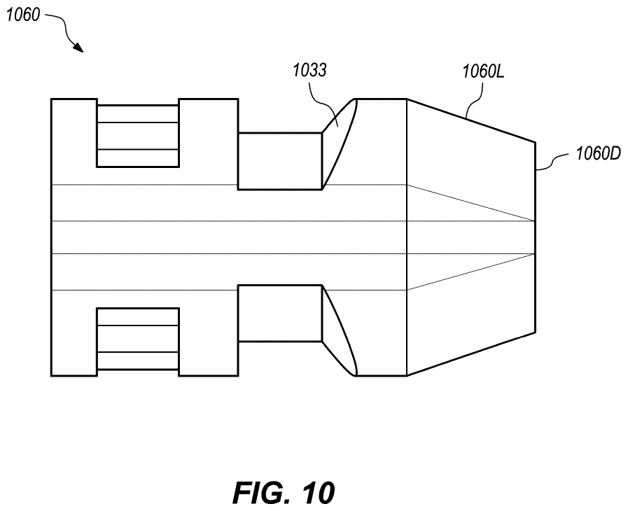

For example, FIG. 10 is a simplified schematic side view illustration of yet another embodiment of the station housing 1060 that can be included as part of one of the emitter stations in any of the embodiments illustrated and described herein above. As shown in FIG. 10, the station housing 1060 includes a leading edge 1060L that is slanted, or tapered, such that the overall cross-section of the station housing 1060 is somewhat narrowed at a housing distal end 1060D. More particularly, the station housing 1060 and/or the leading edge 1060L is slanted, or tapered so as to have a lower profile beyond the plasma target(s) 1033 that are incorporated within the station housing 1060.

With such design, when the balloon 104 (illustrated in FIG. 1) is in the deflated state to be inserted into the blood vessel, with the balloon 104 being shrunken down around the station housing 1060 and tending to take on a portion of the shape of the station housing 1060, the slanted, or tapered, shape of the leading edge 1060L can help break through the vascular lesions 106A (illustrated in FIG. 1) at the treatment site 106 (illustrated in FIG. 1). It is appreciated, however, that typically only the distal-most station housing 1060 in any given embodiment of the catheter system 100 (illustrated in FIG. 1) would need to have such a modification to the leading edge 1060L of the station housing 1060, as any subsequent, more proximally positioned station housings would not need to help break through the vascular lesions 106A at the treatment site 106.

In summary, as described herein, the various embodiments of the station housing for the emitter stations provide many advantages over previously available catheter systems. For example, by utilizing station housings within the emitted stations such as described in detail herein, the user can realize advantages such as (1) only two station housing components are required for proper placement of eight (or more) individual emitters at a pair of emitter stations within the catheter system, as opposed to the eight that would be required in previously available catheter systems; (2) relative placement for the emitters at each emitter station will be more precise since the station housing is an integrally-formed, single-piece component for incorporating four (or more) emitters at the emitter station; (3) the simplified manufacturing process results in fewer components being rejected for failing to meet specifications; (4) manufacturing and assembly of the components to form the emitter stations is greatly simplified, thus enabling use of lower cost and/or lower skilled product builders; (5) simplification of both manufacturing and assembly can help save the user in terms of both time and money; (6) there is a decreased risk of any emitters becoming dislodged during use of the catheter system; and (7) fewer heat shrink bands (if any) are required to secure the energy guides and/or emitters in desired position.

It should be noted that, as used in this specification and the appended claims, the singular forms “a,” “an,” and “the” include plural referents unless the content and/or context clearly dictates otherwise. It should also be noted that the term “or” is generally employed in its sense including “and/or” unless the content or context clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

It is recognized that the figures shown and described are not necessarily drawn to scale, and that they are provided for ease of reference and understanding, and for relative positioning of the structures.

The headings used herein are provided for consistency with suggestions under 37 CFR 1.77 or otherwise to provide organizational cues. These headings shall not be viewed to limit or characterize the invention(s) set out in any claims that may issue from this disclosure. As an example, a description of a technology in the “Background” is not an admission that technology is prior art to any invention(s) in this disclosure. Neither is the “Summary” or “Abstract” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

It is understood that although a number of different embodiments of the catheter system, the emitter system, and/or the emitter station have been illustrated and described herein, one or more features of any one embodiment can be combined with one or more features of one or more of the other embodiments, provided that such combination satisfies the intent of the present invention.

While a number of exemplary aspects and embodiments of the catheter system, the emitter system, and/or the emitter station have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope, and no limitations are intended to the details of construction or design herein shown.

What is claimed is:

1. A catheter system for treating a treatment site within or adjacent to a vessel wall of a blood vessel, or within or adjacent to a heart valve, within a body of a patient, the catheter system being configured to use a guidewire lumen, the catheter system comprising:
an energy source that generates first energy;
a first energy guide that receives the first energy from the energy source, the first energy guide having a first guide distal end; and
a first emitter station including a first station housing having (a) a first housing base that is configured to be secured at least partially about the guidewire lumen, (b) a first guide retainer that extends away from the first housing base, the first guide retainer being configured to selectively receive and retain the first guide distal end of the first energy guide, and (c) a first corresponding plasma target that is spaced apart from the first guide retainer, the first housing base, the first guide retainer, and the first corresponding plasma target being integrally formed with one another.

2. The catheter system of claim 1, wherein the first housing base is at least partially cylinder-shaped and defines an at least partially cylinder-shaped base aperture that extends therethrough; and wherein the guidewire lumen is configured to be positioned at least partially within the base aperture.

3. The catheter system of claim 1, wherein the first guide retainer is substantially annular-shaped.

4. The catheter system of claim 1, wherein the first guide retainer is formed as a groove that extends radially away from the first housing base.

5. The catheter system of claim 1, wherein the first guide distal end of the first energy guide is configured to be secured within the first guide retainer with an adhesive material.

6. The catheter system of claim 1, further comprising:
a first emitter that is incorporated into the first emitter station, the first emitter including (a) the first guide distal end of the first energy guide that is selectively received and retained within the first guide retainer, and (b) the first corresponding plasma target that is spaced apart from the first guide distal end, the first energy guide being configured to emit the first energy in a direction away from the first guide distal end and toward the first corresponding plasma target so that a plasma is generated at the first corresponding plasma target upon receiving the first energy from the first energy guide.

7. The catheter system of claim 1, wherein the energy source further generates second energy; and further comprising a second energy guide that receives the second energy from the energy source, the second energy guide having a second guide distal end.

8. The catheter system of claim 1, wherein the energy source further generates second energy; and further comprising a second energy guide that receives the second energy from the energy source, the second energy guide having a second guide distal end; and a second emitter station including a second station housing having (a) a second housing base that is configured to be secured at least partially about the guidewire lumen, (b) a second guide retainer that extends away from the second housing base, the second guide retainer being configured to selectively receive and retain the second guide distal end of the second energy guide, and (c) a second corresponding plasma target that is spaced apart from the second guide retainer, the second housing base, the second guide retainer, and the second corresponding plasma target being integrally formed with one another.

9. The catheter system of claim 1, wherein the energy source is a laser and the first energy guide is an optical fiber.

10. A catheter system for treating a treatment site within or adjacent to a vessel wall of a blood vessel, or within or adjacent to a heart valve, within a body of a patient, the catheter system comprising:
a plurality of energy guides that each selectively receive at least a portion of the energy from the energy source, each of the plurality of energy guides having a guide distal end;
a first emitter station including a first station housing having (a) a first housing base, (b) a plurality of first guide retainers that extend away from the first housing base, each of the plurality of first guide retainers being configured to selectively receive and retain the guide distal end of one of the plurality of energy guides, and (c) a plurality of first corresponding plasma targets that are each positioned spaced apart from one of the plurality of first guide retainers, the first housing base, the plurality of first guide retainers, and the plurality of first corresponding plasma targets being integrally formed with one another.

11. The catheter system of claim 10, wherein the first housing base is at least partially cylinder-shaped and defines an at least partially cylinder-shaped base aperture that extends therethrough; and wherein a guidewire lumen is configured to be positioned at least partially within the base aperture.

12. The catheter system of claim 10, wherein the first guide retainer is formed as a groove that extends radially away from the first housing base.

13. The catheter system of claim, 10 wherein the guide distal end of at least one of the plurality of first energy guides is configured to be secured within one of the plurality of first guide retainers with an adhesive material.

14. The catheter system of claim 10, further comprising:
a plurality of first emitters that are incorporated into the first emitter station, each of the plurality of first emitters including (a) the guide distal end of one of the plurality of energy guides that is selectively received and retained within one of the plurality of guide retainers, and (b) one of the plurality of corresponding plasma targets that is spaced apart from the guide distal end, each of the plurality of energy guides being configured to emit the at least a portion of the energy from the energy source in a direction away from the guide distal end and toward the corresponding plasma target so that a plasma is generated at the corresponding plasma target upon receiving the at least a portion of the energy from the energy guide.

15. The catheter system of claim 10, further comprising:
a second emitter station including a second station housing having (a) a second housing base, (b) a plurality of second guide retainers that extend away from the second housing base, each of the plurality of second guide retainers being configured to selectively receive and retain the guide distal end of one of the plurality of energy guides, and (c) a plurality of second corresponding plasma targets that are each positioned spaced apart from one of the plurality of second guide retainers, the second housing base, the plurality of second guide retainers, and the plurality of second corresponding plasma targets being integrally formed with one another.

16. A catheter, the catheter comprising:
a guidewire lumen;
a plurality of energy guides that each selectively receive at least a portion of the energy from the energy source, each of the plurality of energy guides having a guide distal end;
a first emitter station positioned around the guidewire lumen, the first emitter station including a first station housing having (a) a first housing base, (b) a plurality of first guide retainers that extend away from the first housing base, each of the plurality of first guide retainers being configured to selectively receive and retain the guide distal end of one of the plurality of energy guides, and (c) a plurality of first corresponding plasma targets that are each positioned spaced apart from one of the plurality of first guide retainers, the first housing base, the plurality of first guide retainers, and the plurality of first corresponding plasma targets being integrally formed with one another.

17. The catheter of claim 16, wherein the first housing base is at least partially cylinder-shaped and defines an at least partially cylinder-shaped base aperture that extends therethrough, and
wherein the guidewire lumen is configured to be positioned at least partially within the base aperture.

18. The catheter of claim 16 wherein the guide distal end of at least one of the plurality of first energy guides is configured to be secured within one of the plurality of first guide retainers with an adhesive material.

19. The catheter of claim 16, further comprising:
a plurality of first emitters that are incorporated into the first emitter station, each of the plurality of first emitters including (a) the guide distal end of one of the plurality of energy guides that is selectively received and retained within one of the plurality of guide retainers, and (b) one of the plurality of corresponding plasma targets that is spaced apart from the guide distal end, each of the plurality of energy guides being configured to emit the at least a portion of the energy from the energy source in a direction away from the guide distal end and toward the corresponding plasma target so that a plasma is generated at the corresponding plasma target upon receiving the at least a portion of the energy from the energy guide.

20. The catheter of claim 16, further comprising:
a second emitter station including a second station housing having (a) a second housing base, (b) a plurality of second guide retainers that extend away from the second housing base, each of the plurality of second guide retainers being configured to selectively receive and retain the guide distal end of one of the plurality of energy guides, and (c) a plurality of second corresponding plasma targets that are each positioned spaced apart from one of the plurality of second guide retainers, the second housing base, the plurality of second guide retainers, and the plurality of second corresponding plasma targets being integrally formed with one another.
